(19) United States
(12) Patent Application Publication
(10) Pub. No.: US 2002/0019051 A1
(43) Pub. Date: Feb. 14, 2002

(54) Chimeric adenoviral vectors

(76) Inventors: Monika Lusky, Freiburg (DE); Arend Jan Winter, Strasbourg (FR)

Correspondence Address: BURNS DOANE SWECKER & MATHIS L L P, POST OFFICE BOX 1404, ALEXANDRIA, VA, 22313-1404, US

(21) Appl. No.: 09/867,475

(22) Filed: May 31, 2001

Related U.S. Application Data
(63) Continuation-in-part of application No. 09/463,486, filed on Jan. 27, 2000.

(30) Foreign Application Priority Data
May 27, 1998 (FR) 98/06654
May 27, 1999 (FR) PCT/FR99/01238

(51) Int. Cl. C12N 15/861; C12N 7/01

(52) U.S. Cl. 435/457; 435/235.1

(57) ABSTRACT

The invention concerns adenoviral vectors having the characteristic of containing a region essential for heterologous packaging with respect to the adenoviral genome from which they are derived. The invention also concerns a method for making a viral preparation containing said adenoviral vectors, a cell, a pharmaceutical composition or material comprising them and their therapeutic or prophylactic use. Finally, the invention concerns an adenoviral genome of animal origin having attenuated packaging properties with respect to the native genome from which it is derived.

The present invention relates to novel adenoviral vectors which have the characteristic of containing a region essential for encapsidation which is heterologous with respect to the adenoviral genome from which they are derived.

These vectors may be used as helper or recombinant vectors, the former allowing propagation of the latter. The subject of the invention is also a method for preparing a viral preparation containing said adenoviral vectors, a cell, a pharmaceutical composition or a composition of material comprising them as well as their use for therapeutic or prophylactic purposes. The present invention also provides a method for producing conventional recombinant minimal vectors using a helper virus containing a heterologous encapsidation region. Finally, the present invention also relates to an adenoviral genome of animal origin having attenuated encapsidation capacities compared with the native genome from which it is derived. The invention is of especial interest in the perspectives of gene therapy, especially in humans.

Gene therapy is defined as the transfer of genetic information into a host cell or organism. The first protocol applied to humans was initiated in the United States in September 1990 on a patient who was genetically immunodeficient because of a mutation affecting the gene encoding Adenine Deaminase (ADA). The relative success of this first experiment encouraged the development of this technology for various diseases including both genetic (with the aim of correcting the dysfunction of a defective gene) and acquired (cancers, infectious diseases such as AIDS and the like) diseases. Most of the current strategies use vectors to carry the therapeutic gene to its cellular target. Many vectors including viral and synthetic vectors have been developed during the past few years and have been the subject of many publications accessible to persons skilled in the art.

The importance of adenoviruses as gene therapy vectors has already been mentioned in many prior art documents. They infect many cell types, both dividing and quiescent cells, are nonintegrative and not very pathogenic. In addition, they possess a natural tropism for the respiratory tracts. These specific properties make adenoviruses vectors of choice for many therapeutic and even vaccine applications. As a guide, their genome consists of a linear and double-stranded DNA molecule of about 36 kb which carries about thirty genes involved in the viral cycle. The early genes (E1 to E4; E for early) are divided into 4 regions dispersed in the genome. The E1, E2 and E4 regions are essential for viral replication whereas the E3 region, which is involved in modulating the antiadenovirus immune response in the host, is not. The E1 region (E1A and E1B) encodes proteins responsible for the regulation of transcription of the viral genome.

Expression of the E2 region genes (E2A and E2B) leads to the synthesis of the polypeptides needed for viral replication, including pTP (pre-Terninal Protein), pol (polymerase) and DBP (DNA Binding Protein) (Pettersson and Roberts, 1986, In Cancer Cells (Vol 4): DNA Tumor Viruses, Botchan and Glodzicker Sharp Eds pp 37-47, Cold Spring Harbor Laboratory, Cold Spring Harbor, N.Y.). The proteins encoded by the E3 region prevent cytolysis by cytotoxic T cells and tumor necrosis factor (Wold and Gooding, 1991, Virology 184, 1-8). The proteins encoded by the E4 region are involved in DNA replication, late gene expression and splicing and host cell shut off (Halbert et al., 1985, J. Virol. 56, 250-257).

The late genes (L1 to L5; L for late) encode predominantly the structural proteins, including the majority of the viral capsid proteins and partially cover the early transcription units. They are for the most part transcribed from the Major Late Promoter (MLP). In addition, the adenoviral genome carries, at its ends, cis-acting regions which are essential for encapsidation, consisting of inverted terminal repeats (ITR) situated at the 5′ and 3′ ends and an encapsidation region which follows the 5′ ITR. The ITRs harbor origins of DNA replication whereas the encapsidation region is required for the packaging of adenoviral DNA into infectious particles. The viral DNA is associated with four polypeptides, namely V, VII, m and terminal protein (TP). The 55 kDa TP is covalently linked to the 5′ ends of the DNA via a dCMP (Rekosh et al., 1977, Cell 11, 283-295; Robinson et al., 1973, Virology 56, 54-69). The other three polypeptides are noncovalently bound to the DNA and fold it in such a way as to fit into the small volume of the capsid.

The adenoviral vectors which are currently used in gene therapy protocols lack the major part of the E1 region in order to avoid their dissemination in the environment and in the host organism. Additional deletions in the E3 region make it possible to increase the cloning capacities. The genes of interest are introduced into the viral DNA in place of one or other of the deleted regions.

While the feasibility of transferring genes using these so-called first-generation vectors is now well established, the question of their safety remains. In addition to the risk of generating replication-competent particles, the potential immunogenecity of the viral proteins still expressed can, in some specific applications, prevent the persistence of the transduced cells and the stable expression of the transgene. These disadvantages have justified the construction of new-generation vectors. They conserve the regions in cis (ITRs and encapsidation sequences) which are essential for encapsidation but comprise additional genetic modifications aimed at suppressing the in vivo expression of most of the viral genes (see for example International Application WO 94/28152). In this regard, a so-called minimal vector, which is deficient for all the adenoviral functions, represents an alternative of choice.

The techniques for preparing adenoviral vectors are widely described in the literature. In a first instance, the genome is prepared by homologous recombination in the 293 line (see in particular Graham and Prevec, 1991, Methods in Molecular Biology, Vol 7, Gene Transfer and Expression Protocols; Ed E. J. Murray, The Human Press Inc, Clinton, N.J.) or in Escherichia coli (see for example International Application WO 96/17070). It is then necessary to propagate the vector in order to constitute a stock of viral particles containing it. This production step is critical and should make it possible to obtain high infectious particle titers to be able to envisage a large-scale development for the purpose of the preparation of clinical batches. Complementation lines providing in trans the viral products of expression for which the vector is defective are used to this effect. For example, the viruses deleted for E1 can be propagated in the 293 line which is established from human embryonic kidney cells (Graham et al., 1977, J. Gen. Virol. 36, 59-72). As regards the second-generation vectors, it is possible to use lines complementing two essential viral functions, such as those described by Yeh et al. (1996, J. Virol. 70, 559-565), Krougliak and Graham (1995, Human Gene Therapy 6, 1575-1586), Wang et al. (1995 Gene Therapy 2, 775-783), Lusky et al. (1998, J. Virol. 72, 2022-2033) and in International Applications WO 94/28152 and WO 97/04119. Because of the potential toxicity of the viral products of expression, these lines need to be optimized in terms of growth capacity and viral particle yield before envisaging their use in an industrial process. Furthermore, a line complementing all the adenoviral functions, suitable for the propagation of the minimal vectors is currently not available.

Another alternative is based on the use of an additional viral element designated “helper virus” to complement, at least in part, the defective functions of a recombinant adenoviral vector. The helper viruses of the prior art consist of an adenoviral genome, optionally deleted for an essential region for which the recombinant vector does not require complementation. By way of example, cotransfection into the 293 line of an E1-helper virus and of an E1-E4recombinant adenoviral vector leads to the formation of viral particles of recombinant vector. The E1 function is provided by the 293 line and the E4 function by the helper virus.

However, a major disadvantage of this method is that the cells produce a mixed population of viral particles, some comprising the recombinant vector and others the helper vector. In practice, the preparations predominantly contain helper viral particles, these having a selective advantage, such that the contamination may reach and even exceed 90%. The presence of the helper virus is not desirable in the context of a therapy applied to humans and, because of this, requires the use of physical separation techniques, such as ultracentrifugation.

Recently, a Cre/loxP helper-dependent system has been described in WO98/13510 and WO97/47757. In this approach, the helper virus is engineered to have its packaging signal flanked by loxP sites that can be recognized by the sitespecific recombinase “Cre”. Recombination catalyzed by Cre will result in the excision of the helper sequences present between the two loxP sites. Thus, upon infection of a 293 cell line that constitutively expresses the Cre recombinase, the packaging signal is excised from the helper virus rendering it unpackageable. However, the helper virus DNA is able to replicate and provide all of the functions necessary in trans for the packaging of a recombinant minimal vector. However, it has been observed that a significant amount of helper virus DNA escapes the Cre-mediated excision event and can therefore be packaged into infectious virions wich contaminate the recombinant viral preparation. The present invention proposes exploiting the respective growth properties of the human and animal adenoviruses. The inability of bovine BAV3 adenoviruses to be propagated in a human line has now been demonstrated whereas Ad5 can be propagated in bovine cells. Indeed, the infection by BAV3 adenoviruses, alone or in the presence of Ad5, in the human 293 line does not lead to the formation of infectious BAV3 viral particles. On the other hand, Ad5 virions are obtained by infecting a bovine line. In addition, there is no expression of BAV3 viral proteins in human cells.

On the basis of these observations, the present invention proposes in particular a system of encapsidation occurring in two stages and using adenoviruses which are chimeras between human (Ad5) and animal (BAV3) adenoviruses. The vector system proposed in the present invention is based on the use of two helper adenovirus constructs and two amplification steps carried out in two different cell lines, respectively a non-human and a human cell line to prepare a stock of a recombinant minimal adenovirus vector. The first adenovirus helper construct is an animal (e.g. BAV3) genome, which replicates well in animal (e.g. bovine) cells and serves as a helper virus for the production of a second (e.g. Ad5) helper from which the endogenous encapsidation region is replaced by a heterologous encapsidation region, from the first helper. This makes packaging of second (Ad5) helper dependent on the presence of the first (BAV3) helper which will provide the packaging functions. The production of the recombinant minimal vector can be made according to two different approaches.

The first approach is outlined in FIG. 1 and relies on the use of a minimal vector engineered to contain two encapsidation regions to be recognized by the packaging machinery from the first and second helper constructs (Ad5 as well as BAV3). The two helpers and the recombinant minimal vector are first amplified in animal (e.g. bovine) cells to produce a mixture of the three different viruses which is used to infect a cell line from human origin. The helper constructs are unable to package since they both contain an encapsidation region from an animal adenovirus and will be eliminated while allowing the amplification of the minimal virus.

There has now been constructed (i) a helper vector derived from an Ad5 genome in which the native encapsidation region is replaced by that of the bovine BAV3 adenovirus and (ii) a recombinant defective adenoviral vector derived from an Ad5 and comprising two encapsidation regions, the first of Ad5 origin (autologous) and the second of BAV3 origin (heterologous). The transfection of the two vectors into a bovine cell line infected with a BAV3 adenovirus leads to the amplification of the three viral genomes and to the production of viral particles of the three types. During this first amplification stage, the BAV3 genome provides the in trans-acting factors allowing the encapsidation of the recombinant and helper vectors and the latter at least partially complements the defective functions of the recombinant vector. The mixture of the three types of virus is recovered from the bovine cells and used to infect human 293 cells. The BAV3 genome and the helper vector possessing only one encapsidation region derived from BAV3 cannot be propagated in the human line even in the presence of Ad5 because of the absence of the encapsidation factors recognizing the BAV3 sequences, which excludes the formation of corresponding viral particles. However, the helper vector can produce in trans the factors necessary for the encapsidation of the recombinant vector mediated by the encapsidation signal of Ad5 origin and complement, in association with the 293 cells, the defective early and late functions, with the aim of predominantly generating virions containing the recombinant vector.

The second alternative (outlined in FIG. 2) can be used to produce a conventional recombinant minimal vector which contains only the Ad5 ITRs and encapsidation region together with the therapeutic gene to be transfered. Contrary to the first approach, the bovine cells are not used for the production of the recombinant minimal vector, but used exclusively for the production of the Ad5 helper in the presence of the bovine BAV3 helper. Then, human cells are transfected with the minimal vector and infected with the virus mixture produced in bovine cells. As with the first alternative, BAV3 helper will not amplify, nor be packaged in human cells and will thus not appear in the final virus preparation. Ad5 helper is also impaired for packaging due to the absence of the Ad5 encapsidation sequences and will be eliminated as well. The conventional minimal vector will be amplified and produced, hopefully without any helper contamination.

This second alternative has the advantage to allow the production of any conventional minimal vector which is carried out exclusively in human cells. Moreover, the amplification step in each cell line utilizes only two different constructs that makes parameters much easier to control (BAV3 helper is not counted for in human cells as it is essentially inactive).

The present invention meets safety objectives by considerably reducing the contamination of the adenoviral preparations by the helper vectors and thus avoids the use of long and expensive separation techniques of varying efficiency.

Accordingly, the subject of the present invention is an adenoviral vector derived from an adenoviral genome, characterized in that it comprises a region essential for encapsidation (also refered in the literature or hereinafter as packaging signal, packaging sequence, packaging region or psi) which is heterologous with respect to the adenoviral genome from which it is derived.

For the purposes of the present invention, an adenoviral vector is obtained from a parental adenovirus whose genome is modified. A minimal modification is the insertion of a region essential for encapsidation of a different origin (heterologous). Of course, other modifications may also be envisaged. These may be of various types (deletion, addition, substitution of one or more nucleotides) and may be located in coding regions of the adenoviral genome or outside these (regions involved in the expression of the viral genes, in the encapsidation and the like) and may be affected both in the early and late regions. In this regard, an adenoviral vector which is particularly suitable for the present invention is defective, that is to say is incapable of being autonomously propagated in a host cell in the absence of complementation. It may be defective for one or more viral genes which are essential for replication. These genes may be deleted (as a whole or in part), made nonfunctional (for example by mutation) or substituted by other sequences (in particular by a gene of interest whose expression is sought in a host cell or organism).

The adenoviral vector of the present invention may be derived from a human or animal adenovirus and of any serotype. The subgroup C human adenoviruses and in particular the adenoviruses 2 (Ad2) and 5 (Ad5) are most particularly suitable for carrying out the invention. Among the animal adenoviruses which can be used in the context of the present invention, there may be mentioned canine, avian, bovine, murine, ovine, porcine and simian adenoviruses and the like. As a guide, it is possible to use the murine adenoviruses Mavl (Beard et al., 1990, Virology 175, 81-90), the canine adenoviruses CAV-1 or CAV-2 (Spibey and Cavanagh, J. Gen. Virol., 1989, 70, 165-172; Linné, 1992, Virus Research 23, 119-133; Shibata et al., 1989, Virol. 172, 460-467; Jouvenne et al., Gene, 1987, 60, 21-28), the avian adenoviruses DAV (Zakharchuk et al., Arch. Virol., 1993, 128, 171-176) or the bovine adenoviruses BAV3 (Mittal et al., J. Gen. Virol., 1995, 76, 93-102). In general, the abovementioned adenoviruses are available in collections and in particular at the ATCC and have been the subject of many studies published in the prior art. As regards adenovirus 5 (Ad5), it should be noted that the complete sequence of its genome is available from GenBank under accession number M73260. This sequence is fully incorporated by reference into the present application.

For the purposes of the present invention, “region essential for encapsidation” is understood to mean a region acting in cis to ensure, in collaboration with protein factors, in particular viral protein factors, the encapsidation of a viral vector genome into a viral capsid. Such regions consist in particular, in the case of the adenoviral genome, of the encapsidation (or packaging) region and, optionally the 5′ and 3′ ITRs. These terms are well known in the field of the art considered.

The characteristic of the adenoviral vector according to the invention is that it carries a region essential for encapsidation which is heterologous, that is to say of a different origin, with respect to the parental adenovirus. Although it may be derived from any virus (retrovirus, poxyirus and the like), an adenoviral origin is preferred as long as it is an adenovirus of a genus or serotype which is different from the parental adenovirus. Preferably, according to the present invention, said heterologous region essential for encapsidation consists of the encapsidation sequence and optionally of at least one of the 5′ and 3′ ITRs.

Depending on the origin of the adenovirus, the regions essential for encapsidation may vary somewhat. However, they can be identified on the basis of the available sequence and literature data or by analogy with human adenoviruses. The ITRs are naturally located at the 5′ and 3′ ends of the adenoviral genome and are involved in the stages of replication and encapsidation of said genome. Generally, the ITRs comprise between 100 and 200 base pairs. Numerous ITR sequences are proposed in the literature; there may be mentioned by way of example Hearing et al., 1987, J. Virol., 61, 2555-2558 for Ad5 or WO 95/16048 for BAV3. The encapsidation region (noted T or psi) is located behind the 5′ ITR of the adenoviral genome and comprises repetitive motifs which participate in the encapsidation. For example, that of Ad5 comprises 7 motifs designated Al to AVII having the consensus sequence 5′ A/T AN A/T TTTG 3′ (where N represents any nucleotide) and situated at positions 241-248, 262-269, 304-311, 314-321 and 3395 346 of the viral genome (Grable and Hearing, 1990, J. Virol. 64, 2047-2056; Schmid and Hearing, 1998, J. Virol. 72, 6339-6347) or TTTGN₈CG (where N represents any nucleotide ; Schmid et al. 1998, J. Virol. 72, 6339-6347). The encapsidation regions of various adenoviruses are described in the literature (see for example Hammarskjold and Winberg, 1980, Cell 20, 787-795 for Adl6; Hearing et al., 1987, J. Virol. 61, 2555-2558 for Ad5; Robinson and Tibbets, 1984, Virology, 137, 276-286 for Ad3; Shibata et al., 1989, Virology 172, 460-467 for CAV2; WO 95/16048 for BAV3). Purely by way of illustration, it should be mentioned that the encapsidation region of Ad5 extends at least from the nucleotides (nt) 240 to 350. As for that of BAV3, it is contained in a fragment of 0.3 kb between the positions about 185 to about 514. However, the limits of the encapsidation region can vary and shorter or longer regions are also suitable. Persons skilled in the art are capable of isolating a fragment of the 5′ end of an adenoviral genome, of inserting it into an appropriate vector and of verifying its encapsidation capacities in an appropriate line, for example by determining the viral titer or the expression of a reporter gene.

The sequences carrying the heterologous region essential for encapsidation may be isolated from a viral genome by conventional means (digestion with a restriction enzyme, PCR and the like) or may be produced by chemical synthesis. Optionally, in the context of the present invention, they may comprise mutations (deletion, substitution and/or addition of one or more nucleotides) compared with the native sequences. It is also possible to include other exogenous sequences (restriction sites and the like). They may be inserted into the adenoviral vector according to the invention in addition to the autologous region or as a replacement thereof. The insertion may take place in 5′ or in 3′ of the autologous region, in its place or at a different site (for example at the 3′ end just before the 3′ ITR).

Advantageously, the adenoviral vector according to the invention is derived from an adenovirus of human origin and the heterologous region essential for encapsidation from an adenovirus of animal origin. In this regard, a vector which is most particularly suitable for the present invention is derived from a subgroup C human adenovirus and, in particular from an adenovirus 2 (Ad2) or 5 (Ad5). As for the heterologous region essential for encapsidation, it is preferably derived from an animal adenovirus selected from those mentioned above.

According to a completely preferred embodiment, the adenoviral vector according to the invention is derived from an Ad5 and the heterologous region essential for encapsidation from a bovine adenovirus, in particular from a BAV3.

It should be mentioned that the abovementioned embodiments are preferred but that other combinations may be used in the context of the present invention. It is possible, for example, to envisage an adenoviral vector derived from an AdS and comprising a heterologous region essential for encapsidation derived from another human adenovirus of a different serotype (Ad3, Ad7 and the like). Alternatively, the adenoviral skeleton may be of animal origin and the region essential for encapsidation derived from a human adenovirus.

As indicated above, the adenoviral vector according to the invention is preferably defective at least for the E1 function. Such a deficiency may be obtained by mutation or total or partial deletion of the corresponding region. Many E1 vectors are described in the prior art and may be used in the context of the present invention. In addition, it may comprise additional mutations/deletions affecting one or more other viral genes, in particular in the E2, E4 and/or L1-L5 regions. Any combination may be envisaged (E1⁻ E2⁻, E1⁻ E4⁻, E1⁻ E2⁻ E4⁻ and the like). Such vectors are in particular described in International Application WO 94/28152. To illustrate these embodiments, there may be mentioned the heat-sensitive mutation affecting the DBP (for DNA Binding Protein) gene of the E2A region (Ensinger et al., 1972, J. Virol. 10, 328-339). A partial deletion of the E4 region, with the exception of the sequences encoding the open reading frames (ORF) 6 and 7, may also be envisaged (Ketner et al., 1989, Nucleic Acids Res. 17, 3037-3048). Another possibility is the total deletion of the transcriptional unit E4. Moreover, the adenoviral vector according to the invention may lack all or part of the nonessential region E3. According to this alternative, it may be advantageous to conserve, nevertheless, the E3 sequences encoding the polypeptides allowing escape from the immune system of the host, in particular the glycoprotein gp19k (Gooding et al., 1990, Critical Review of Immunology 10, 53-71). In some applications (recombinant vector), the nonfunctionality of all the viral genes is preferred.

According to a first variant, the adenoviral vector according to the invention may be used as helper viral vector to complement all or part of the defective functions of a recombinant adenoviral vector. According to an advantageous embodiment, it is defective at least for the E1 function. Optionally, it may be defective for additional functions such as E2. Persons skilled in the art are capable of defining the required deficiencies depending on the recombinant vector which it is sought to complement and the chosen cell line. It should be mentioned that the E4 function may be provided by only ORF 6 and 7. The presence of all or part of the E3 region is optional. According to a specific embodiment, it comprises the 5′ and 3′ ITR sequences and the sequences encoding the E2, E4 and/or L1-L5 functions derived from a human adenovirus, in particular from an Ad5, and a heterologous encapsidation region derived from a bovine adenovirus, in particular from a BAV3. According to another embodiment, the helper viral vector comprises the sequences encoding the E2, E4 and/or L1-L5 functions derived from a human adenovirus, in particular from an Ad5 and heterologous 5′ ITR sequences, 3′ ITR sequences and encapsidation region which are derived from a bovine adenovirus, in particular from a BAV3.

A helper adenoviral vector according to the invention is obtained by insertion, into an adenoviral genome as defined above, of at least one heterologous region essential for encapsidation. A preferred manner of proceeding is to replace the autologous region essential for encapsidation with the heterologous region.

Persons skilled in the art are capable of producing such a construct by applying conventional molecular biology techniques.

According to a second variant, the adenoviral vector according to the invention is a recombinant adenoviral vector and comprises at least one gene of interest placed under the control of elements necessary for its expression in a host cell or organism.

The gene of interest used in the present invention may be derived from a eukaryotic organism, from a prokaryote, from a parasite or from a virus other than an adenovirus. It may be isolated by any technique conventionally used in the field of the art, for example by cloning, PCR or chemical synthesis. It may be of the genomic type (comprising all or part of the introns as a whole), of the complementary DNA type (cDNA, lacking introns) or of the mixed type (minigene). Moreover, it may encode an antisense RNA and/or a messenger RNA (mRNA) which will then be translated into a polypeptide of interest, it being possible for the latter to be (i) intracellular, (ii) incorporated into the membrane of the host cell or (iii) secreted. It may be a polypeptide as found in nature (native), a portion thereof (truncated), a mutant exhibiting in particular improved or modified biological properties or alternatively a chimeric polypeptide obtained from the fusion of sequences of various origins.

Within the context of the present invention, it may be advantageous to use a gene of interest which encodes a cytokine α, β or γ interferon, interleukin (IL), in particular IL-2, IL-6, IL-10 or IL-12, a tumor necrosis factor (TNF), a colony-stimulating factor (GM-CSF, C-CSF, M-CSF, etc.), a cell receptor (in particular recognized by the HIV virus), a receptor ligand, a coagulation factor, a growth factor (FGF, standing for Fibroblast Growth Factor, VEGF, standing for Vascular Endothelial Growth Factor), an enzyme (urease, renin, thrombin, metalloproteinase, NOS, standing for Nitric Oxide Synthetase, SOD, catalase, etc.), an enzyme inhibitor (α1-antitrypsin, antithrombin III, viral protease inhibitor, PAI-1, standing for plasminogen activator inhibitor), a class I or II major histocompatibility complex antigen or a polypeptide which acts on the expression of the corresponding genes, a polypeptide which is able to inhibit a viral, bacterial or parasitic infection or its development, a polypeptide which reacts positively or negatively on apoptosis (Bax, Bc12, Bc1X, etc.), a cytostatic agent (p21, p16, Rb), an apolipoprotein (ApoAI, ApoAIV, ApoE, etc.), an angiogenesis inhibitor (angiostatin, endostatin, etc.), a marker β-galactosidase, luciferase, etc.) or any other gene of interest which has a therapeutic effect on the targeted ailment. More precisely, for the purpose of treating an hereditary malfunction, use will be made of a functional copy of the defective gene, for example a gene encoding factor VIII or IX within the context of A or B hemophilia, dystrophin within the context of Duchenne's and Becker's myopathies, insulin within the context of diabetes, and the CFTR (Cystic Fibrosis Transmembrane Conductance Regulator) protein within the context of cystic fibrosis. As regards inhibiting the initiation or progress of tumors or cancers, preference will be given to using a gene of interest which encodes an antisense RNA, a ribozyme, a cytotoxic product (herpes simplex virus 1 thymidine kinase (HSV-1-TK), ricin, cholera or diphtheria toxin, product of the FCY1 and FUR1 yeast genes encoding uracil phosphoribosyl transferase and cytosine deaminase, etc.), an antibody, an inhibitor of cell division or transduction signals, an expression product of a tumor suppressor gene (p53, Rb, p73, etc.), a polypeptide which stimulates the immune system, a tumor-associated antigen (MUC-1, BRCA-1, early or late antigens (E6, E7, LI, L2, etc.) of an HPV papilloma virus, etc.) where appropriate in combination with a cytokine gene. Finally, use can be made, within the context of an anti-HIV therapy, of a gene which encodes an immunoprotective polypeptide, an antigenic epitope, an antibody (2F5; Buchacher et al., 1992, Vaccines 92, 191-195), the extracellular domain of the CD4 receptor (sCD4; Traunecker et al., 1988, Nature 331, 84-86), an immunoadhesin (for example a CD4-IgG immunoglobulin hybrid; Capon et al., 1989, Nature 337, 525-531; Byrn et al., 1990, Nature 344, 667-670), an immunotoxin (for example fusion of the antibody 2F5 or the immunoadhesin CD42F5 to angiogenin; Kurachi et al., 1985, Biochemistry 24, 5494-5499), a transdominant variant (EP 0614980, WO 95/16780), a cytotoxic product such as one of those mentioned above, or else an α or β IFN.

Moreover, one of the genes of interest may also be a selectable gene which makes it possible to select or identify the transfected or transduced cells. There may be mentioned the neo gene (encoding neomycin phosphotransferase), which confers resistance to the antibiotic G418, the dhfr (Dihydrofolate Reductase) gene, the CAT (Chloramphenicol Acetyl Transferase) gene, the pac (Puromycin Acetyl Transferase) gene or the gpt (Xanthine Guanine Phosphoribosyl Transferase) gene. In general, the selectable genes are known to persons skilled in the art.

The phrase “elements necessary for the expression” designates the genetic elements allowing the transcription of a gene of interest into RNA and the translation of an mRNA into a polypeptide. Among these, the promoter is of particular importance. It may be isolated from any gene of eukaryotic or even viral origin and may be constitutive or regulatable. Alternatively, it may be the natural promoter of the gene in question. Moreover, it may be modified so as to improve the promoter activity, suppress a region which inhibits transcription, make a constitutive promoter regulatable or vice versa, introduce a restriction site and the like. There may be mentioned, by way of examples, the eukaryotic promoters of the PGK (Phospho Glycerate Kinase), MT (metallothionein; Mclvor et al., 1987, Mol. Cell. Biol. 7, 838-848), al-antitrypsin, CFTR, surfactant, immunoglobulin, β-actin (Tabin et al., 1982, Mol. Cell. Biol. 2, 426-436) or SRα (Takebe et al., 1988, Mol. Cell. Biol. 8, 466-472) genes, the SV40 virus (Simian Virus) early promoter, the RSV (Rous Sarcoma Virus) LTR, the HSV-1-TK promoter, the CMV virus (Cytomegalovirus) early promoter and the adenoviral promoters E1A and MLP. It may also be a promoter which stimulates expression in a tumor or cancer cell. There may be mentioned in particular the promoters of the MUC-1 gene which is overexpressed in breast and prostate cancers (Chen et al., 1995, J. Clin. Invest. 96, 2775-2782), the CEA (for carcinoma embryonic antigen) gene which is overexpressed in colon cancers (Schrewe et al., 1990, Mol. Cell. Biol. 10, 2738-2748), the tyrosinase gene which is overexpressed in melanomas (Vile et al., 1993, Cancer Res. 53, 3860-3864), the ERB-2 gene which is overexpressed in cancers of the breast and of the pancreas (Harris et al., 1994, Gene Therapy 1, 170-175) and the α-fetoprotein gene which is overexpressed in liver cancers (Kanai et al., 1997, Cancer Res. 57, 461-465). The Cytomegalovirus (CMV) early promoter is most particularly preferred.

The elements necessary for the expression may, in addition, include additional elements which improve the expression of the gene of interest or its maintenance in the host cell. There may be mentioned in particular the intron sequences, signal sequences for secretion, nuclear localization sequences, internal sites for reinitiation of translation of the IRES type, poly A sequences for termination of transcription, tripartite leaders and replication origins. These elements are known to persons skilled in the art.

When the recombinant adenoviral vector according to the invention comprises several genes of interest, these may be placed under the control of the same genetic elements (polycistronic cassette using an internal site for initiation of translation of the IRES type for reinitiating the translation of the second cistron) or of independent elements. The independent expression cassettes may be inserted in the same location of the adenoviral backbone (e.g. in replacement of any deleted adenoviral region) or in different locations (e.g. in replacement of the E1 and E3 deleted regions) in the same or opposite directions.

A particularly advantageous embodiment consists in a recombinant adenoviral vector comprising a second region essential for encapsidation which is autologous with respect to the adenoviral genome from which it is derived. In other words, it carries two encapsidation regions, one autologous and the other heterologous. Their position in the adenoviral vector is unimportant. They may be placed in particular at one of the ends or separated at each of the ends of said vector. Advantageously, the heterologous region is placed in 3′ of the autologous region.

Within the context of the present invention, a recombinant adenoviral vector according to the invention is defective for the E1 function and at least one of the E2, E4 and/or L1-L5 functions. A preferred example is provided by a vector defective for all the adenoviral functions (minimum vector) which comprises, in addition to the gene(s) of interest, at least the 5′ and 3′ ITRs and an autologous region essential for encapsidation derived from a human adenovirus, in particular from an Ad5 and a heterologous region essential for encapsidation derived from a bovine adenovirus, in particular from a BAV3.

It is within the capability of persons skilled in the art to generate a recombinant adenoviral vector according to the invention by molecular biology techniques. They will obviously know how to adapt the technology as a function of the specific data (type of vector, gene of interest and the like).

The present invention also encompasses vectors or viral particles that have been modified to allow preferential targeting of a particular target cell. A characteristic feature of targeted vectors/particles is the presence at their surface of a targeting moiety capable of recognizing and binding to a cellular and surface-exposed component. Such targeting moieties include without limitation chemical conjugates, lipids, glycolipids, hormones, sugars, polymers (e.g. PEG, polylysine, PEI and the like), peptides, polypeptides (for example JTS1 as described in WO 94/40958), oligonucleotides, vitamins, antigens, lectins, antibodies and fragments thereof. They are preferably capable of recognizing and binding to cell-specific markers, tissue-specific markers, cellular receptors, viral antigens, antigenic epitopes or tumor-associated markers.

The specificity of infection of adenoviruses is determined by the attachment to cellular receptors present at the surface of permissive cells. In this regard, the fiber and penton present at the surface of the adenoviral capsid play a critical role in cellular attachment (Defer et al., 1990, J. Virol. 64, 3661-3673). Thus, cell targeting of adenoviruses can be carried out by genetic modification of the viral gene encoding fiber and/or penton, to generate modified fiber and/or penton capable of specific interaction with unique cell surface polypeptides. Examples of such modifications are described in literature (for example in Wickam et al., 1997, J. Virol. 71, 8221-8229; Arnberg et al., 1997, Virol. 227, 239-244; Michael et al., 1995, Gene Therapy 2, 660-668; WO94/10323). To illustrate, inserting a sequence coding for EGF within the sequence encoding the adenoviral fiber will allow to target EGF receptor expressing cells. Other methods for cell specific targeting may be achieved by the chemical conjugation of ligands (e.g. antibodies or antibody fragments) to the adenoviral surface.

The present invention also relates to the use of a helper adenoviral vector according to the invention for complementing all or part of the defective functions of a replication-defective recombinant adenoviral vector, in particular of a conventional minimal (also named gutless) recombinant adenoviral vector or a recombinant adenoviral vector according to the invention. The helper and recombinant vectors preferably both derive from the same adenovirus and, in particular from an Ad5, and said heterologous regions essential for encapsidation carried by the helper vector and, optionally, the recombinant vector derive from the same adenovirus different from the preceding one and in particular from a BAV3.

The present invention also relates to a composition comprising:

(a) a helper adenoviral vector according to the invention,

(b) a replication-defective recombinant adenoviral vector, in particular a recombinant adenoviral vector according to the invention, and

(c) optionally, an adenoviral genome which is derived from an animal adenovirus of the same origin as the heterologous regions essential for encapsidation carried by the helper and recombinant vectors a) and optionally b).

The present invention also relates to an adenoviral genome which is derived from an animal adenovirus, in particular from a bovine adenovirus and, in particular, from a BAV3, characterized in that it has an attenuated encapsidation capacity compared with the adenovirus from which it is derived. The attenuation is intended to reduce the propagation of the adenoviral genome to the benefit of a genome carrying a native region (helper and recombinant adenoviral vector according to the invention). It may be obtained by partial deletion of the encapsidation region or by mutation of one or more motifs controlling the encapsidation process. An example of attenuation is provided in International Application WO 94/28152 and in Imler et al. (1995, Human Gene Therapy 6, 711-721). Persons skilled in the art know the techniques which make it possible to verify the attenuation, for example by determining the viral titer (Graham and Prevec, 1991, supra) or the expression of a reporter gene with respect to an equivalent virus carrying a native region essential for encapsidation. An attenuated region essential for encapsidation exhibits an encapsidation efficiency which is reduced by a factor of 2 to 1000, advantageously of 3 to 100 and, preferably, of 5 to 50.

The animal adenoviral genome according to the invention may be replication-competent or may comprise modifications affecting one or more viral genes, such as those cited above. In particular, the total or partial deletion of the E1 region of said genome may be advantageous within the context of the present invention. It should be mentioned that the genome and/or the vectors may be in the form of DNA or of a virus.

The present invention also relates to a method for preparing a viral preparation comprising a replication-defective recombinant adenoviral vector, in particular a recombinant adenoviral vector according to the invention, according to which:

(a) there are introduced into a first cell line

(i) a helper adenoviral vector according to the invention,

(ii) an adenoviral genome which is derived from an animal adenovirus, and

(iii) said recombinant adenoviral vector,

said adenoviral genome (ii) being of the same origin as the heterologous regions essential for encapsidation which are carried by the vectors (i) and optionally (iii) and said adenoviral genome (ii) and the vectors (i) and (iii) being capable of replicating in said first cell line,

(b) said first cell line is cultured under appropriate conditions to allow the production of viral particles comprising the vectors (i) and (iii) and the adenoviral genome (ii),

(c) said viral particles obtained in step b) are recovered from the cell culture,

(d) a second cell line is infected with said viral particles recovered in step c), said helper vector (i) and said adenoviral genome (ii) having a zero or reduced encapsidation and/or replication capacity in said second cell line,

(e) said second cell line is cultured under appropriate conditions to allow the encapsidation of said recombinant adenoviral vector (iii) and produce said viral preparation, and

(f) said viral preparation obtained in step e) is recovered from the cell culture.

For the purposes of the present invention, the adenoviral vectors and genomes may be introduced by any prior art means into the first cell line, in particular by transfection and/or infection. It is possible to transfect the vectors into the line which is infected with particles of animal adenoviral genome (prior to, subsequent to or concomitant with the transfection). The viruses containing the various elements (i), (ii) or (iii) may be prepared according to prior art techniques. Moreover, said replication-defective recombinant adenoviral vector may consist of a vector as described in WO 94/28152, WO 94/08026, WO 931/19191 or WO 94/12649.

Moreover, the genome (ii) may be of the wild type, according to the invention (attenuated) and/or comprise one or more modifications affecting the functionality of one or more viral genes.

According to an advantageous embodiment, the first cell comprises

(i) a helper adenoviral vector which is defective at least for the E1 function, derived from a human adenovirus (in particular from an Ad5) and carrying a heterologous region essential for encapsidation derived from a bovine adenovirus (in particular from a BAV3),

(ii) an adenoviral genome derived from a bovine adenovirus (in particular from a BAV3), optionally according to the invention, and

(iii) a recombinant adenoviral vector which is defective for the E1 function and at least one of the E2, E4 and/or L1-L5 functions and carrying a second autologous encapsidation region as defined above, derived from a human adenovirus (in particular from an AdS) and carrying a heterologous region essential for encapsidation derived from a bovine adenovirus (in particular from a BAV3), said first cell line being of bovine origin.

According to a completely advantageous embodiment, the defective helper adenoviral vector in question in (i) of the methods described above comprises a heterologous region essential for encapsidation derived from a bovine adenovirus (in particular from a BAV3) which comprises the 5′ and 3′ ITRs and the encapsidation region.

Alternatively, it is possible to use an adenoviral genome (ii) which is defective for the E1 function. As regards the preferred variant, a BAV3 genome which is defective for the E1 function is described in International Application WO 95/16048. In this case, use will be made of a first cell line capable of complementing the E1 function of said adenoviral genome of animal origin. A line of the same animal origin as said adenoviral genome (ii) will be preferably used. It may be an established line or a primary line.

The term complementation cell is standard in the field of the art. Within the context of the present invention, it refers to a eukaryotic cell capable of providing in trans at least part of the defective functions of an adenoviral vector or genome according to the invention. In general, a cell for complementing an adenoviral function may be obtained by transfecting the corresponding viral genes into an appropriate cell line. All the standard means for introducing a DNA into a cell may be used (transfection with calcium phosphate, electroporation, microinjection, lipofection, protoplast fusion and the like). Moreover, the viral genes are carried by conventional vectors (synthetic, viral or plasmid vectors and the like) and placed under the control of elements allowing their constitutive or regulated expression in said complementation cell. The complementation lines appropriate for the adenoviral vectors are known to persons skilled in the art (see for example International Application WO 94/28152, WO 97/04119 and Graham et al., J. Gen. Virol., 1977, 36: 59-72).

A particularly suitable bovine line is derived from an MDBK line (ATCC CCL-22 or CRL-6071) or from primary cells, in particular of the retina or of fetal kidney, and comprises the sequences encoding the E1 region of a bovine adenovirus, and in particular of a BAV3, which are placed under the control of the elements necessary for their expression in said line.

According to a preferred embodiment, the second cell line is a cell for complementing the E1 function of a human adenovirus, in particular of an Ad5. Use will be preferably made of the 293 line. However, other lines such as those described in Application WO 94/28152 may also be used.

The viral particles of step c) and the viral preparation may be recovered from the culture supernatant but also from the cells. One of the methods commonly used consists in lysing the cells by successive freeze/thaw cycles in order to recover the virions from the lysis supernatant. These may then be amplified and purified according to prior art techniques (chromatographic method, ultracentrifugation in particular through a cesium chloride gradient, and the like).

The present invention also covers a second alternative method for preparing a recombinant minimal adenoviral vector stock comprising:

(a) Introducing in a first cell line (i) a first helper adenoviral vector or virus and (ii) a second helper adenoviral vector or virus the genome of (i) and (ii) comprising 5′ and 3′ ITRs, an encapsidation region and one or more gene(s) of the early and/or late regions,

The genome of (i) deriving from a first adenovirus genome,

The genome of (ii) deriving from a second adenovirus genome different from said first adenovirus with the exception of at least the region essential for encapsidation which derives from said first adenovirus genome,

Said first helper (i) being capable of packaging said second helper (ii) in said first cell line;

(b) culturing the cell obtained in step (a) under appropriate conditions to allow the production of viral particles comprising (ii) and, optionnally (i),

(c) recovering the viral particles obtained in step (b) from the cell culture,

(d) introducing in a second cell line said viral particles obtained in step (c) and a recombinant minimal vector,

(e) culturing the cell obtained in step (d) under appropriate conditions to allow the production of viral particles comprising said recombinant minimal vector, and

(f) recovering the viral particles obtained in step (e) from the cell culture.

The method of the present invention is outlined in FIG. 2. It requires two amplification steps, the first one carried out in a first cell line to amplify and produce the helper viruses (steps a to c) and the second one carried out in a second cell line in the presence of the helper viruses to amplify and produce the viral preparation of interest of recombinant minimal viruses.

One indicates that the term “vector” is intended to cover both DNA vector and a viral particle (virion or virus) The first and second helper constructs (i) and (ii) are essentially as described before. The first and second adenovirus may differ from each other in terms of serotypes or origins. Preferably, the first adenovirus is an animal adenovirus and especially a bovine adenovirus whereas the second adenovirus is a human adenovirus. In one preferred embodiment, the first adenovirus is BAV3 and the second adenovirus is Ad5.

The term “deriving” intends to cover the embodiment according to which the first and/or second helper adenoviral vector(s) that are used in the context of the method of the present invention is (are) a wild-type adenovirus genome(s) or include(s) modification(s) thereof. Preferably the modification(s) that are introduced in the wild type genome lead(s) to a defective mutant altered in one or more function(s) as described above. The functional defect in one or the other helper can be obtained by any kind of modification(s) in the adenoviral genome (deletion, addition, substitution of one or more nucleotides in one or more genes of one or more adenoviral region) and can affect an early region and/or a late region, a coding sequence and/or a sequence involved in the transcriptional or translational regulation of the concerned region. When the modification impairs the replication and/or packaging of one or both helpers in the first cell line, it is required that the defective function be complemented at least partially either by the other helper adenoviral vector or by the first cell line. By way of illustration, the first helper adenoviral vector is defective in E1 function and the defective E1 gene products can be provided by using either an E1+ second adenoviral helper vector (as ilustrated in the examples, Ad5 E1 can complement for E1 BAV3 function) or by a E1 complementation cell line (e.g. VIDO R2 described in WO00/26395). In one particularly embodiment, the first and second helper adenoviral vectors are defective mutants of wild-type adenovirus genomes and are capable of cross-complementing each other for at least one defective function. Preferably, the first helper is defective for E1 function. However, a mutant defective in any other adenoviral function(s), (E2, E3, E4, L1-L5 or any combination thereof) can also be used. For example, one may contemplate the use of a first helper adenoviral vector defective in E2 function. Preferably, said defective E2 function is caused by a mutation or deletion in at least the gene encoding DBP, Pol and/or pTP.

The second helper adenoviral vector is characterized by an heterologous encapsidation region as described before. As mentioned before, the heterologous encapsidation region can be inserted anywhere in the genome of said second adenoviral helper vector but preferred insertion site includes downstream of the ITR5′ or upstream of the ITR 3′, with a special preference for insertion directly downstream of the ITR5′.

As mentioned before, the genome of the second helper adenoviral vector may be a wild-type adenovirus genome or a mutant defective for any early and/or late function or any combination thereof. A special preference is a second helper adenoviral vector defective for E1 and, optionally, E3 function. A preferred second adenoviral heper vector is an Ad5 genome deleted of nucleotides approximately 455 to approximately 3327 (deletion of the E1 region) and having nucleotides approximately 149 to approximately 454 comprising the Ad5 encapsidation region replaced by nucleotides approximately 141 to approximately 984 of the BAV3 genome (pTG13327).

According to an advantageous embodiment, the second adenoviral vector used in the context of the present invention may further comprise all or part of a second encapsidation region which preferably derives from said second adenovirus genome (endogenous) in addition of the heterologous one. The additional (endogenous) encapsidation region can be present at its original location or be inserted at any location within the second helper genome, for example just upstream of the 3′ ITR. This particular embodiment allows to increase packaging efficiency of the second (Ad5) helper with respect to the first (BAV3) in the first cell line.

This embodiment applies more specifically to E1+ second adenoviral helper vector due to the overlap of the E1 promoter element with the endogenous encapsidation region. To ensure efficient expression of the E1 genes from the E1 promoter, it is possible to reintroduce upstream of the E1 region at least the portion of the adenoviral genome that contain the E1 promoter elements. It is however preferred to ensure E1 expression in said second helper by placing the E1 region under the control of a non-adenoviral promoter. In this consideration, an advantageous embodiment relates to a second adenoviral vector which is functional for the E1 function, wherein the E1 region is placed under the control of a non-adenoviral promoter. Any eukaryotic or viral promoter is suitable in the context of the present invention, such as the PGK promoter.

According to a particularly advantageous embodiment, both the first and second helper adenoviral vectors have an origin of replication recognized by the same E2 encoded gene products. As illustrated in FIG. 7, the adenoviral 5′ and 3′ ITRs harbor the origin of replication which covers the penultimate 50 bp of the ITRs. This region contain more specifically the core origin (within the penultimate 20bp) which is recognized by the E2-encoded proteins pTP, DBP and pol. The binding of these adenoviral proteins is essential to initiate replication. The core origin is followed by a so-called “auxiliary region” containing binding sites of cellular nuclear factors (NFI, oct-1 also named NFIII) which acts to enhance replication of the viral genome. As illustrated in the examples, the E2 BAV3 products are unable to bind the core origin of Ad5 ITRs and initiate replication of an Ad5 viral genome. Therefore, in the context of the present invention, one may consider to modifiy either the core region (present in the penultimate 20 bp of adenoviral ITRs), the entire origin of replication (contained in the penultimate 50 bp of adenoviral ITRs) or the entire ITRs of one helper to allow binding of the E2 gene products expressed by the other helper adenoviral vector.

According to one variant, the endogenous 5′ and 3′ ITRs of the first adenoviral helper vector are modified to make the origin of replication recognized by the E2 gene products expressed from the second adenoviral helper vector. The modification may consist in the replacement of the penultimate 20 bp containing the core region, the penultimate 50 bp containing the entire origin of replication or the entire ITRs of said first adenoviral helper vector by the equivalent sequence of the 5′ and 3′ ITRs of said second adenoviral helper vector or any functional equivalent thereof (modified compared to the wild type sequence but capable of providing binding of the viral and cellular factors and initiating replication of the adenoviral genome). By way of illustration, a proposed modification consists in the replacement of the C present in position 14 of the BAV3 ITRs in A as found in the same position of Ad5 ITRs and the A present in position 18 of the BAV3 ITRs in C as found in the same position of Ad5 ITRs. One may also replace the endogenous 5′ and 3′ ITRs of the first helper by the 5′ and 3′ ITRs of the second helper adenoviral vector. In this respect, a preferred example is a BAV3 helper adenoviral vector supplied with Ad5 ITRs. This embodiment has the advantage to prevent or minimize potentially deleterious recombination events between the first and second helper adenoviral vectors that may lead to unsignificant production of the required helper by the first cell line. The first adenoviral helper vector is preferably deficient (mutated or deleted) in the E2 region to avoid expression of the endogenous E2 gene products pTP, pol and/or DBP.

According to a second variant of this embodiment, it is also possible to modify the 5′ and 3′ ITRs of the second helper adenoviral vector to make the origin of replication present on said ITRs recognized by the E2 gene products expressed from the first adenoviral helper vector. The same kind of modifications as described above can be considered, for example the mutation of the core and/or auxiliary of the origin of replication or the replacement of the endogenous 5′ and 3′ ITRs of said second helper by those of (or deriving from) the first adenovirus.

In this consideration, a preferred second helper adenoviral vector is obtained from an Ad5 genome deleted of nucleotides approximately 455 to approximately 3327 (deletion of the E1 region) and nucleotides approximately 28592 to approximately 30470 (deletion of the E3 region) and having nucleotides approximately 1 to approximately 454 comprising the ITR 5′ and the Ad5 encapsidation region replaced by nucleotides approximately 1 to approximately 984 of the BAV3 genome and nucleotides approximately 35826 to approximately 35935 comprising the ITR 3′ replaced by nucleotides approximately 34188 to approximately 34446 of the BAV3 genome (pTG14316).

The first cell line used in the method of the present invention is as defined before. It is advantageously a non-human cell line, preferably from bovine origin (especially when the first adenoviral helper vector is or derives from a BAV3 genome) although other mammalian cell lines can also be contemplated. A suitable first cell line may also be obtained from cotton rat lung fibroblasts.

In one embodiment, the first cell line is capable of complementing part or all of at least one defective function of said first or second or first and second helper(s). In particular, it can be advantageous to use a first cell line capable of complementing the E1 function of said first or second or both first and second adenoviral helper vector(s). Suitable cell lines include Ad5 E1 complementing cell lines such as VIDO R2 obtained by introducing a plasmid vector encoding the Ad5 E1 region in a primary culture of fetal bovine retina cells as described in WO00/26395.

The helper constructs are introduced into the first cell line according to any delivery technique of the art. One may proceed via viral transduction using infectious viral particles or by transfection of the DNA vectors into the first cell line. The transfection technique can be performed using any standard protocol, such as calcium phosphate precipitation, DEAE-dextran, electroporation, direct microinjection, DNA-loaded liposomes, cell sonication, gene bombardement and receptor-mediated transfection. The first and second helpers can be introduced in the same time (co-transduction or co-infection) or independently, the order having no importance using the same introduction technique or different techniques (e.g. transfection by calcium phosphate precipitation of one helper and viral transduction of the other helper).

As indicated before, the method of the present invention comprises a second amplification step carried out in a second cell line (steps (d) to (f) in which the viral mixture obtained from the first cell line is used to amplify and produce a recombinant replication-deficient adenoviral vector.

The recombinant replication-deficient adenoviral vector is as defined above. It includes one or more gene(s) of interest under the control the elements necessary for its (their) expression in a host cell or organism, as described before.

The recombinant replication-deficient adenoviral vector is preferably a so-called “minimal vector” or “gutless vector” which is completely devoid of viral coding sequences and only contain the cis-acting sequences that are found at the extremities of the adenoviral genome, namely the 5′ and 3′ ITRs (involved in the replication of the viral genome) and the encapsidation region (which allows the viral DNA to be packaged) located just downstream of the 5′ ITR. Conventional minimal vectors are described in the literature accessible to the man skilled in the art (see for example WO94/28152). Preferably, the minimal vector derives from the Ad5 genome in which the 5′ ITR and encapsidation region together comprise approximately the first 400 to 500 bp present at the 5′ extremity of the Ad5 genome and the 3′ ITR comprise the last 100 to 150 bp present at the 3′ extremity of the Ad5 genome. The design of the minimal vector itself is relatively simple. Apart from the cis-acting sequences described above, which are prerequisites for the replication of the viral genome and its packaging in viral capsids, there is only a size requirement. The minimum size of the viral genome to allow an afficient encapsidation is 75% of the wild type size (36 kb) or 27 kb. The maximum size that can be packaged is approximately 4% above the wild type genome size or 37,5 kb. This means a total insert size of 26,5 to 37 kb. Since most expression cassettes are much smaller than 26,5 kb, it is advantageous to introduce DNA sequences that make up for the difference. The so-called stuffer sequences can be any origin, including prokaryotic, but eukaryotic origin is preferred to avoid or minimize a possible immune response in the host cell or organism. Indeed, prokaryotic stuffer sequences may contain cryptic promoters that may induce expression of potentially immunogenic (poly)peptides. Preferably, the stuffer sequences originate from eukaryotic DNA that is not expressed, i.e. intergenic sequences or sequences derived from known genes from which the promoter has been deleted. According to a preferred embodiment, the method of the invention utilizes stuffer DNA derived from the human gene coding for blood clotting factor IX (hFIX) (as described for example in WO91/2056 and U.S. Pat. No. 5,814,716). To facilitate the cloning of a variety of expression cassettes, the minimal vector backbone can be modified in order to introduce suitable restriction sites or multiple cloning sites.

In one embodiment, the recombinant minimal vector can contain an additional encapsidation region to improve its packaging in viral capsid. The two encapsidation regions have the same origin and are preferably identical (e.g. from Ad5). They can be inserted in the same location of the recombinant vector (e.g. just after the 5′ ITR in head to tail orientation) or at different location (e.g., just after the 5′ ITR and just just upstream of the 3′ ITR)

According to the method of the invention, the second cell line is as defined above. It is preferably of human origin. In one embodiment, it is capable of complementing part or all of at least one defective function of said recombinant minimal vector and, more particularly the Ad5 E1 function. Suitable second cell lines include without any limitation, 293 cells (Graham et al., 1977, J. Gen. Virol. 36, 59-72) and PER.C6 (Fallaux et al., 1998, Human Gene Ther. 9, 1909-1917).

Although any transduction and transfection techniques may be used to introduce said helpers and minimal vector in the second cell line (see above), it is preferable to first transfect the minimal vector construct in the cell line which is then infected by the viral helper mixture obtained from step (c).

The method of the present invention may comprise more than one amplification step, particularly in the second cell line. For example, the resultant viral particles obtained in step (f) are used to reinfect said second cell line, preferably, in the presence of fresh second adenoviral helper vector or virus.

The method of the present invention may further comprises a purification step of the viral particles obtained in step (f). A number of different purification techniques may be employed, including clarification, lysis, enzymatic treatment, filtration, ultrafiltration, ultracentrifugation and chromatography or any combination thereof. Such a purification protocol is described in more detail for example in WO96/27677, WO97/08298, WO98/00524, WO98/22588, WO98/26048, WO00/40702 and WO00/50573.

The method of the present invention is intended to provide viral particles of minimal vector as obtained in step (f) substantially free of helper viruses.

The present invention also relates to an animal adenovirus genome having modified 5′ and 3′ ITRs and wherein said modification consists in the replacement of:

the penultimate 20 bp containing the core origin,

the penultimate 50 bp containing the entire origin of replication or

the entire ITRs

of said animal adenovirus genome by the homologous sequences of the 5′ and 3′ ITRs of a human adenovirus genome. Such modifications are detailed hereinabove. In addition, the animal adenovirus genome of the present invention can comprise additional modifications of its genome, such as those detailed above (for example altering at least one of the early and/or late function(s)).

The present invention also relates to the viral preparation obtained according to the method according to the invention. According to an advantageous embodiment, it comprises at least 30% of infectious viral particles containing the recombinant adenoviral vector according to the invention. Advantageously, it comprises at least 50%, preferably at least 70%, and most preferably at least 80% of said particles.

The present invention also relates to a host cell comprising an adenoviral vector according to the invention or infected with a viral preparation according to the invention. A mammalian cell, and in particular a human cell, is most particularly suitable. It may comprise said vector in a form integrated into the genome or otherwise (episome). It may be a primary or tumor cell of a hematopoietic origin (totipotent stem cell, leukocyte, lymphocyte, monocyte or macrophage and the like), muscle cell (satellite cell, myocyte, myoblast and the like), cardiac, pulmonary, tracheal, nasal, hepatic, vascular, arterial, renal, epithelial, endothelial or fibroblast cell.

The present invention also relates to a cell comprising:

(i) a helper adenoviral vector according to the invention,

(ii) an adenoviral genome which is derived from an animal adenovirus, and

(iii) a replication-defective recombinant adenoviral vector, in particular a recombinant adenoviral vector according to the invention.

Said cell is preferably a cell for complementing an adenoviral function and, in particular, the E1 function of an animal or human adenovirus. It has the characteristics defined above.

The present invention also relates to a pharmaceutical composition comprising, as therapeutic or prophylactic agent, an adenoviral vector, a viral preparation or a host cell according to the invention in combination with a pharmaceutically acceptable carrier. The composition according to the invention is more particularly intended for the preventive or curative treatment of diseases by gene therapy and applies to both genetic diseases (hemophilia, diabetes, cystic fibrosis, Duchenne's or Becker's myopathy, autoimmune diseases) and acquired diseases (cancers, tumors, cardiovascular diseases, diseases of infectious origin such as hepatitis B or C, AIDS and the like).

A pharmaceutical composition according to the invention may be manufactured in a conventional manner for administration by the local, parenteral or digestive route. In particular, a therapeutically effective quantity of the therapeutic or prophylactic agent is combined with a pharmaceutically acceptable carrier. The routes of administration which may be envisaged are many. There may be mentioned, for example, the intragastric, subcutaneous, intracardiac, intramuscular, intravenous, intraarterial, intraperitoneal, intratumor, intranasal, intrapulmonary or intratracheal route. For the latter three embodiments, administration by aerosol or instillation is advantageous. The administration may be made as a single dose or as a dose which is repeated once or several times after a certain time interval. The appropriate route of administration and dosage vary as a function of various parameters, for example the individual or the disease to be treated or the gene(s) of interest to be transferred. The viral preparation according to the invention may be formulated in the form of doses of between 10⁴ and 10¹⁴ pfu (plaque forming units), advantageously 10⁵ and 10¹³ pfu, and preferably 10⁶ and 10¹² pfu. As regards the recombinant adenoviral vector according to the invention, doses comprising 0.01 to 100 mg of DNA, preferably 0.05 to 10 mg and most preferably 0.5 to 5 mg may be envisaged. The formulation may also include a pharmaceutically acceptable diluent, adjuvant or excipient. It may be provided in liquid or dry form (lyophylizate and the like).

The viral vector or preparation according to the invention may be optionally combined with one or more substances which improve the transfection efficiency and/or the stability. These substances are widely documented in the literature which is accessible to persons skilled in the art (see, for example, Felgner et al., 1987, Proc. West. Pharmacol. Soc. 32, 115-121; Hodgson and Solaiman, 1996, Nature Biotechnology 14, 339-342; Remy et al., 1994, Bioconjugate Chemistry 5, 647-654). By way of nonlimiting illustration, they may be polymers, lipids, in particular cationic lipids, liposomes, nuclear proteins or neutral lipids, nuclease inhibitors, hydrogel, hyaluronidase (WO98/53853), collagenase, polymers, chelating agents (EP890362), in order to preserve its degradation within the animal/human body and/or improve delivery into the host cell. These substances may be used alone or in combination. (e.g. cationic and neutral lipids). It may also comprise substances susceptible to facilitate gene transfer for special applications, such as a gel complex of polylysine and lactose facilitating delivery by intraarterial route (Midoux et al., 1993, Nucleic Acid Res. 21, 871-878) or poloxamer 407 (Pastore, 1994, Circulation 90, 1-517). It has also be shown that adenovirus proteins are capable of destabilizing endosomes and enhancing the uptake of DNA into cells. The mixture of adenoviruses to solutions containing a lipid-complexed plasmid vector or the binding of DNA to polylysine covalently attached to adenoviruses using protein cross-linking agents may substantially improve the uptake and expression of the vector (Curiel et al., 1992, Am. J. Respir. Cell. Mol. Biol. 6, 247-252).

In a preferred embodiment, the composition comprises a pharmaceutically acceptable carrier, allowing its use in a method for the therapeutic treatment of humans or animals. In this particular case, the carrier is preferably a pharmaceutically suitable injectable carrier or diluent which is non-toxic to a human or animal organism at the dosage and concentration employed (for examples, see Remington's Pharmaceutical Sciences, 16^{th} ed. 1980, Mack Publishing Co). It is preferably isotonic, hypotonic or weakly hypertonic and has a relatively low ionic strength, such as provided by a sucrose solution. Furthermore, it may contain any relevant solvents, aqueous or partly aqueous liquid carriers comprising sterile, pyrogen-free water, dispersion media, coatings, and equivalents, or diluents (e.g. Tris-HCl, acetate, phosphate), emulsifiers, solubilizers, excipients or adjuvants. The pH of the composition is suitably adjusted and buffered in order to be appropriate for use in humans or animals. Representative examples of carriers or diluents for an injectable composition include water, isotonic saline solutions which are preferably buffered at a physiological pH (such as phosphate buffered saline, Tris buffered saline, mannitol, dextrose, glycerol containing or not polypeptides or proteins such as human serum albumin). For example, such a composition may comprise 10 mg/ml mannitol, 1 mg/ml HSA, 20 mM Tris pH 7.2 and 150 mM NaCl. Finally, the present invention relates to the use of an adenoviral vector, of a viral preparation or of a host cell according to the invention for the transfer and the expression of a gene of interest in a host cell or organism. A preferred use consists in the treatment of the human or animal body by gene therapy or immunotherapy. According to a first possibility, the medicament may be administered directly in vivo (for example by intravenous injection into an accessible tumor, into the lungs by aerosol and the like). It is also possible to adopt the ex vivo approach which consists in collecting cells from the patient (bone marrow stem cells, peripheral blood lymphocytes, muscle cells and the like), transfecting or infecting them in vitro according to prior art techniques and readministering them to the patient. The preferred use is for the preparation of a medicament intended for the treatment of diseases by gene therapy or immunotherapy. Within the scope of the present invention, “gene therapy” has to be understood as a method for introducing any expressible sequence into a cell. Thus, it also includes immunotherapy that relates to the introduction of a potentially antigenic epitope into a cell to induce an immune response which can be cellular or humoral or both.

The invention also extends to a method of treatment according to which a therapeutically effective quantity of a recombinant adenoviral vector, of a viral preparation or of a host cell according to the invention is administered to a patient requiring such a treatment.

A <<therapeutically effective quantity>> is a dose sufficient for the alleviation of one or more symptoms normally associated with the disease or condition desired to be treated. When prophylactic use is concerned, this term means a dose sufficient to prevent or to delay the establishment of a disease or condition. The present invention is particularly intended for the preventive or curative treatment of disorders, conditions or diseases associated with blood vessels (preferably arteries) and/or the cardiovascular system, including without limitation hypertension, atherogenesis, intimal hyperplasia, (re)restenosis following angioplasty or stent placement, ischemia, neoplastic diseases (e.g. tumors and tumor metastasis), benign tumors, connective tissue disorders (e.g. rheumatoid arthritis, atherosclerosis), ocular angiogenic diseases (e.g. diabetic retinopathy, macular degeneration, corneal graft rejection, neovascular glaucoma), cardiovascular diseases, cerebral vascular diseases, diabetes-associated diseases and immune disorders (e.g. chronic inflammation or autoimmunity). The present invention is also appropriate for the treatment or prevention of any condition or disease caused by infection, cancer and hereditary disorders. The term “cancer” encompasses any cancerous conditions including diffuse or localized tumors, metastasis, cancerous polyps and preneoplastic lesions (e.g. dysplasies) as well as diseases which result from unwanted cell proliferation. One may cite more particularly cancers of breast, cervix (in particular, those induced by a papilloma virus), prostate, lung, bladder, liver, colorectal, pancreas, stomach, esophagus, larynx, central nervous system, blood (lymphomas, leukemia, etc.), melanomas and mastocytoma.

Prevention or treatment of a disease or a condition can be carried out using the present method alone or, if desired, in conjunction with presently available methods (e.g. radiation, chemotherapy and surgery such as angioplasty). Furthermore, in order to improve the transfection rate, the patient may undergo a macrophage depletion treatment prior to administration of the composition of the invention. Such a technique is described in literature (for example in Van Rooijen et al., 1997, TibTech, 15, 178-184).

The invention has been described in an illustrative manner, and it is to be understood that the terminology which has been used is intended to be in the nature of words of description rather than of limitation. Obviously, many modifications and variations of the present invention are possible in light of the above teachings. It is therefore to be understood that within the scope of the appended claims, the invention may be practiced in a different way from what is specifically described herein.

All of the above cited disclosures of patents, publications and database entries are specifically incorporated herein by reference in their entirety to the same extent as if each such individual patent, publication or entry were specifically and individually indicated to be incorporated by reference.

### FIGURES LEGENDS

FIG. 1 illustrates a first alternative to produce recombinant minimal vector using chimeric viruses. ψ represents the encapsidation region of the adenoviral constructs, H means human and B bovine origins.

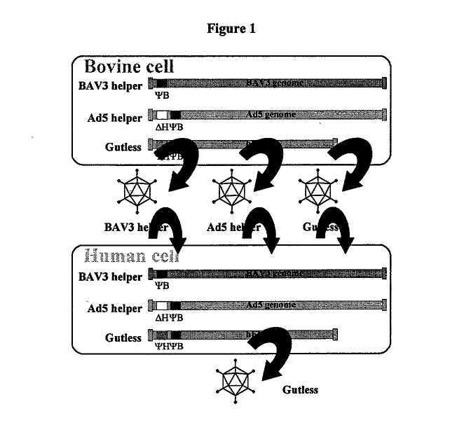

FIG. 2 illustrates a second alternative to produce recombinant minimal vector using chimeric viruses. ψ, H and B are as in FIG. 1.

FIG. 3 illustrates the basic set of Ad5 helper constructs. ψ, H and B are as in FIG. 1.

FIG. 4 illustrates Ad5 helper constructs which exploit complementation of a functional E1 region placed under the control of the endogenous E1 promoter (pTG 14927 and pTG 14929) supplied with either a small bovine encapsidation region (pTG 14927 0,33 kb) or a longer one (pTG 14929 0.84 kb). pTG13366 (small bovine encapsidation region) and pTG 13367 (long bovine encapsidation region) are control constructs without a functional E1 region.

FIG. 5 illustrates Ad5 helper constructs which exploit complementation of a functional E1 region placed under the control of the murine PGK promoter. ITR represents BAV3 inverted terminal repeat sequences, ψ represents bovine encapsidation region (0.84 kb), ψ? represents BAV3 sequence potentially implicated in packaging (0.12 kb) and PGK>E1 represents the Ad5 region driven by the murine PGK promoter.

FIG. 6 illustrates the minimal vector constructs AdTG 14899 and AdTg 15202.

FIG. 7 illustrates the adenoviral origin of replication. The binding sites for the viral pTP-pol complex and cellular factors NFI and Oct-1 are indicated for the Ad5 sequence. The sequence from BAV3 is shown aligned with the sequence from Ad5.

### EXAMPLES

The present invention is illustrated by the following examples without being limited as a result.

The constructs described below are produced according to general genetic engineering and molecular cloning techniques which are detailed in Maniatis et al., (1989, Laboratory Manual, Cold Spring Harbor, Laboratory Press, Cold Spring Harbor, N.Y.) or according to the manufacturer's recommendations when a commercial kit is used. The homologous recombination steps are preferably carried out in the E. coli strain BJ 5183 (Hanahan, 1983, J. Mol. Biol. 166, 557-580). As regards the repair of the restriction sites, the technique used consists in filling the protruding 5′ ends with the aid of the large fragment of DNA polymerase I of E. Coli (Klenow). Moreover, the adenoviral genome fragments used in the various constructs described below are precisely indicated according to their position in the nucleotide sequence of the Ad5 and BAV3 genome, as disclosed in the GeneBank data bank under the reference M73260 and AFO30154 respectively.

As regards the cell biology, the cells are transfected or transduced and cultured according to standard techniques well known to persons skilled in the art. In the examples which follow, use is made of the cell lines 293 (Graham et al., 1977, supra; available from ATCC under the reference CRL1573) and MDBK (ATCC CRL-6071 or CCL-22). It is understood that other cell lines can be used.

### Example 1

Absence of Multiplication of the BAV3 Viruses in the Human lines

The human line MDBK and the human lines A549 (ATCC CCL-1 85), HeLa (ATCC CCL-2) and 293 (ATCC CRL-1573) are infected with a wild-type BAV3 virus at different MOIs (1, 2 and 10). The cells are harvested 3 days after the infection and the viral titers determined on the permissive MDBK cells. The multiplication factor is less than 1 in the case of the infection of the human cells whereas it is between 50 and 100 for the bovine line. These results indicate the inability of the BAV3 vector to be propagated in the human lines.

The expression of the viral genes of BAV3 is verified by reverse PCR after infection of the BAV3 virus into established human lines (A549, HeLa, 293, MRC5, RPMI) or primary human lines (primary muscle line PHM). The control line consists of the MDBK line. The cells are recovered and the polyA+RNAs are isolated according to conventional technique. The reverse transcription is carried out with an antisense primer specific for the E1 region and followed by PCR amplification with the same primer and a sense primer specific for E1. The PCR products are subjected to Southern blot analysis and detected with a probe specific for E1 which makes it possible to detect a band of 626 bp corresponding to the genomic DNA and of 519 bp derived from the polyA+mRNA. In the MDBK cells, an expression of the viral E1 gene which is maximal 16 hours after the infection is observed. E1 is also expressed in the 293 line. On the other hand, no expression of E1 mRNA can be detected in all the other human lines tested.

These results show that the human cells are infected with the BAV3 virus but that the latter cannot replicate therein.

### Example 2

Construction of Chimeric Adenoviral Vectors Ad5/BAV3

A vector is first of all constructed which comprises a cassette for expressing the bacterial LacZ gene encoding β-galactosidase. To do this, the XhoI-SalI fragment of pTG8595 (Lusky et al., 1998, J. Virol. 72, 2022-2033) carrying the LacZ coding sequences is cloned into the XhoI site of the plasmid pCI (Promega). The latter is a eukaryotic expression vector comprising the CMV promoter, splicing sequences, multiple cloning sites and the SV40 polyA sequence. The cassette for expressing the LacZ gene, flanked by the abovementioned regulatory elements, is isolated in the form of a BglII-BamHI fragment and introduced into the BglII site of the transfer vector pTG8343, to give pTG6452. As a guide, the transfer vector comprises the 5′ end of Ad5 deleted for the majority of the E1 sequences (nt 1 to 458 and 3329 to 5788), inserted into a plasmid ppolyII (Lathe et al., 1987, Gene 57, 193-201). The adenoviral genome is reconstituted by homologous recombination between the PacI-NsiI fragment obtained from pTG6452 and the vector pTG3652 carrying the Ad5 genome deleted for the E3 region, cleaved by the enzyme ClaI. The vector thus obtained, called pTG6481, contains the Ad5 sequences lacking the E1 region (nt459 to 3328) and the E3 region (nt 28249 to 30758) and the pCMV-LacZ-pA SV40 cassette cloned in place of the E1 region. The encapsidation region is of Ad5 origin.

The next step is to insert the encapsidation region of BAV3 into the preceding vector. Two sites of insertion were tested: the first upstream of the native encapsidation region, at the level of the AflIII site at position 151 of the Ad5 genome and the other downstream thereof at the level of the SalI site (at position 5 451 of Ad5). The first construct pTG6466 is generated by AflIII digestion of PTG6452, treatment with Klenow and introduction of the HaeII-PvuII fragment of 844 bp covering the sequences at positions 141 to 984 of the BAV3 genome, this fragment having being subjected beforehand to the action of Klenow. The vector pTG6467 is generated according to the same protocol, except that pTG6452 is linearized with SalI. Thus, the vectors pTG6466 and pTG6467 contain two encapsidation regions, one of Ad5 origin and the other of BAV3 origin (about 0.8 kb).

A reduced BAV3 encapsidation region derived from the preceding one by ThaI or BstuI digestion (positions 185 to 514 of the BAV3 genome) was also used. As above, the BAV3 region of about 0.3 kb is inserted into the Ad5 recombinant vectors either at the level of the AflIII site (in 5′ of the autologous psi region) or at the level of the SalI site (in 3′ of the autologous psi region). In the latter case, the transfer vector pTG6458 is generated and the adenoviral genome is reconstituted by homologous recombination as above to give pTG6482.

A construct is then generated in which the Ad5 encapsidation region is exchanged against its BAV3 homologue. The vector pTG6452 is digested with AflIII (nt 151) and SalI (nt 451) and then treated with Klenow polymerase. The fragment of 300bp carrying the Ad5 encapsidation region is replaced with the HaeII-PvuII fragment (844 bp) isolated from BAV3 and made blunt by the action of Klenow. pTG6468 is obtained which carries the sole BAV3 encapsidation region in an Ad5 context. An identical construct uses the BAV3 encapsidation region of 0.3 kb.

The modified regions are reintroduced into the adenoviral genome by homologous recombination as indicated above.

In an identical manner, a helper adenoviral vector derived from the Ad5 genome comprising the encapsidation region and the 5′ and 3′ ITRs of BAV3 was constructed. For that, the vector pTG13373 which is derived from the Ad5 genome deleted for the E1 and E3 regions is constructed, comprising the 5′ ITR and the encapsidation region of 0.3 kb of the BAV3 genome.

The vector pTG 13373 is obtained by replacing the 5′ ITR and the Ad5 encapsidation region which are present in pTG 8343 with the 5′ ITR and the BAV3 encapsidation region which are present in pTG 5431; as a guide, pTG 5431 consists of the 5′ end (nucleotides 1 to 8217) of the BAV3 genome. To do this, pTG 8343 is digested with BglII, subjected to the action of Klenow, and then digested with PacI, and pTG 5431 is digested with AccI, treated with Klenow, and digested with PacI. The fragments thus obtained are linked to give pTG 13372. Finally, pTG 13373 is obtained by homologous recombination between the fragments obtained from pTG 13372, digested with BglI, and from pTG 6401 digested with PacI.

The vector pTG 14310 derived from the Ad5 genome deleted for the E1 and E3 regions and comprising the 5′ and 3′ ITRs as well as the BAV3 encapsidation region is then obtained in the following manner.

The AdS 3′ ITR in the vector pTG 13384 (consisting of the 3′ end (nucleotides 32800 to 35935) of the Ad5 genome; a cloning cassette being inserted between nucleotides 35826 and 35827) is replaced with the BAV3 3′ ITR. To do this, pTG 13384 is digested with XbaI, subjected to the action of Klenow, and then digested with PacI. Moreover, pTG5451 is digested with Apol, treated with Klenow, and digested with PacI. As a guide, the vector pTG 5451 comprises the 5′ end (nucleotides 1 to 1651 deleted for the 829-1077 fragment) and the 3′ end of BAV3 (nucleotides 33232 to 34446) separated by a unique HindIII restriction site.

The fragments thus obtained are linked to give pTG 14261.

Moreover, the recircularization of pTG 14261 after digestion with XbaI and BamHI and action of Klenow allows the elimination of the HindIII cleavage site upstream of the bovine ITR of pTG 14261. The vector pTG 14262 is thus obtained.

In parallel, pTG 13373 digested with HindIII is recircularized in order to obtain pTG 14263 which contains the 5′ ITR and the encapsidation sequence of 0.3 kb of BAV3 as well as the 3′ ITR of Ad5.

The AdS 3′ ITR in the vector pTG 14263 is then replaced with the BAV3 3′ ITR of pTG 14262. To do this, pTG 14263 and pTG 14262 are digested with HindIII and PacI. The fragments thus obtained are linked to give pTG 14266.

Finally, the adenoviral genome is reconstituted by homologous recombination between pTG 14266 linearized with HindlIl and pTG 13373 digested with PacI. The vector obtained, called pTG 14310, is derived from the genome of Ad5 deleted for the E1 and E3 regions and contains the 5′ and 3′ ITRs as well as the BAV3 encapsidation region.

An adenoviral genome similar to that of pTG 14310, but comprising a BAV3 encapsidation region of 0.8 kb, was also obtained. For that, the 5′ ITR and the encapsidation region of Ad5 which are present in pTG 8343 were replaced with the 5′ ITR and the encapsidation region of 0.8 kb of BAV3 which are present in pTG 5431. To do this, pTG 8343 is digested with BglII, subjected to the action of Klenow and then digested with PacI. On the other hand, pTG 5431 is digested with PvuII and PacI. The fragments thus obtained are linked to give pTG 14313.

The 5′ ITR and the encapsidation region of 0.3 kb of BAV3 which are present in pTG 14266 are then replaced with the 5′ ITR and the encapsidation region of 0.8 kb of BAV3 which are present in pTG 14313. To do this, a homologous recombination is carried out between the SspI/MfeI fragment obtained from pTG 14266 and the ScaI/NsiI fragment obtained from pTG 14313. The vector thus obtained is called pTG 14315.

Finally, the adenoviral genome is reconstituted by homologous recombination between the Hindll fragment obtained from pTG 14315 and the PacI fragment obtained from pTG 6401. The vector thus obtained, called pTG 14316, is derived from the Ad5 genome and comprises the 5′ and 3′ ITRs as well as the encapsidation region of 0.8 kb of BAV3.

An additional example of a vector according to the invention consists in a vector which is derived from the AdS genome deleted for the E1 and E3 regions and containing the 5′ and 3′ ITRs as well as the encapsidation region of 0.3 kb of BAV3, in which the encapsidation region has been inserted just upstream of the 3′ ITR.

To construct such a vector, the BAV3 encapsidation region in pTG 13372 and in pTG 14266 is first of all deleted. For that, pTG 13372 is digested with AflIII, subjected to the action of Klenow and then digested with PacI, and pTG 14262 is digested with PvuII and PacI. The fragments thus obtained are linked to give pTG 14311.

The deletion of the bovine encapsidation region of pTG 14266 is obtained by homologous recombination between the XmnI/DraIII fragment obtained from pTG 14266 and the PvuI/SphI fragment obtained from pTG 14311. The vector pTG 14328 is then obtained.

The Ad5 3′ ITR in pTG 13384 is then replaced with the 3′ ITR and the encapsidation sequence of 0.3 kb of BAV3 which are present in pTG 5431. For that, pTG 13384 is digested with BamHI, subjected to the action of Klenow and then digested with PacI. On the other hand, pTG 5431 is digested with AflIII, treated with Klenow and digested with PacI. The fragments thus obtained are linked to give pTG 14271.

The introduction of the BAV3 encapsidation sequence just upstream of the 3′ ITR is carried out by ligation of the EcoRI/HindlIl fragments obtained from pTG14328 and pTG 14271. The vector thus obtained is called pTG 14330.

The adenoviral genome is then reconstituted by homologous recombination between the HindIII fragment of pTG 14330 and the PacI fragment of pTG 6401. The vector pTG prod 11 is then obtained which is derived from the Ad5 genome deleted for the E 1 and E3 regions and contains the 5′ and 3′ ITRs as well as the encapsidation region of 0.3 kb of BAV3 inserted just upstream of the 3′ ITR.

### Example 3

Production of Viral Particles

The viral genomes pTG6468 (helper) and pTG6467 or pTG6466 (recombinant) are transfected into the bovine cells of the MBDK line. Next, the transfected cells are infected with a wild-type or attenuated BAV3 genome. Since Ad5 can be propagated in bovine cells in the presence of a BAV3 virus and since the three viral elements contain a BAV3 encapsidation region, they can be packaged into the viral capsids and generate infectious viral particles. The mixture is recovered and a step of amplification by successive cycles of infection of MBDK cells is optionally carried out so as to constitute a viral stock of the three types of virus. During this first step on bovine cells, the BAV3 genome produces transacting factors for the encapsidation of the helper vector which provides the viral functions necessary for the propagation of the recombinant vector. The encapsidation of the latter can be mediated by adenoviral factors of BAV3 and Ad5 origin since it possesses the encapsidation regions of both origins and the capsids may contain BAV3 or Ad5 structural proteins.

The viral mixture generated in the bovine line is used to infect human 293 cells. In these cells, the BAV3 virus cannot be propagated even in the presence of Ad5. However, the helper vector of Ad5 origin can replicate its viral genome and express all the early and late viral genes which it carries. On the other hand, it cannot be encapsidated since it is equipped with a single BAV3 encapsidation region. By contrast, the recombinant vector can be encapsidated via the encapsidation region of Ad5 origin and the encapsidation factors provided by the helper vector. The viral particles generated are recovered and can be used for therapeutic purposes.

### Example 4

Alternative Strategy for the Production of Minimal Vectors

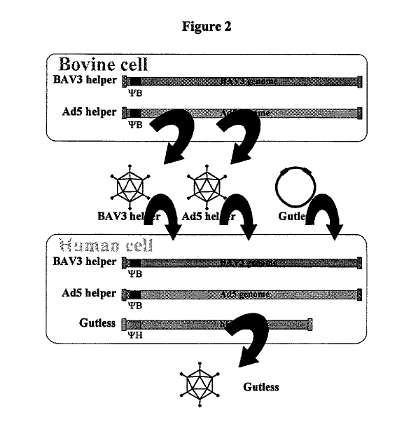

The alternative strategy is outlined in FIG. 2. The bovine cells are used exclusively for the production of Ad5 helper with the help of the bovine helper virus as in Example 3. Ad5 helper is needed for the production of the minimal vector (MV) which is performed in human cell. To start the amplification, MV can be transfected in human cells, which are then super-infected with the Ad5 helper-containing viral mixture produced in bovine cells. As with Example 3, BAV3 helper will not amplify, nor be packaged in human cells and will thus not appear in the final virus preparation. Ad5 helper is also impaired for packaging due to the absence of the Ad5 pacakging sequence and will be eliminated as well. It is likely that serial passages will be needed to increase the minimal vector titers by using the produced minimal virus in cycle 1 as input for cycle 2, etc. Of course, at each cycle fresh Ad5 helper should be added, as this construct is lost with each passage.

A. Materials and Methods

1. Vector Construction

BAV3 E1A°: BAV3 E1A° (also named pFBAV102A) is an EIA deleted BAV3 genome. The deleted region is a PstI-SpeI fragment from position 829 to 1077.

pTG 3602: the full length wild-type Ad5 genome cloned in pPoly II (Chartier et al., 1996, J. Virol. 70, 4805-4810)

pTG 3652: Ad5 genome with a 2.5 kb deletion in the E3 region (position 28249-30751, a BamHI site is inserted in the deletion site) cloned in pPoly II

pTG 3960: plasmid containing the human factor IX gene with a deletion in intron A (PvuII fragment) and a deletion in intron F. A multiple cloning site (MCS) is present in the deletion site in intron F. (Jallat et al., 1990, EMBO J. 9, 3295-3301)

pTG 4671: Expression plasmid for the Ad5 E1A/B region (nt 505 to 4034) driven by the murine PGK promoter. The cassette contains a globin polyadenylation signal downstream of the EIB open reading frame. (Imler et al., 1996, Gene Ther. 3 75-84).

pTG 5435: Wild type BAV3 genome cloned in pPoly II (Rasmussen et al., 1999, Hum. Gen. Ther. 10, 2587-2599; Reddy et al., 1999, J. Virol. 73, 9137-9144)

pTG 6401: described in example 2

pTG 8595: described in example 2

pTG 13327: The Ad5 helper plasmid pTG 13327 contains a 0.8 kb encapsidation signal from BAV3 in place of the encapsidation signal from Ad5. The plasmid is constructed by homologous recombination between ClaI digested pTG 4656 which is an E1/E3 deleted Ad5 with a LacZ expression cassette in place of the E1 region, and pTG 6456 digested with Paci and XmnI. As a guide, transfer plasmid pTG 6456 contains an Ad5 ITR (nt 1-151), flanked at the 3′ end by a 0.8 kb fragment containing the encapsidation signal from BAV3 (a HaeII-PvuII fragment, nt 141-984 from BAV3) followed by nt 33295788 of the Ad5 genome. Thus, during the homologous recombination, the Ad5 packaging element, as well as the LacZ expression cassette in pTG 4656 are replaced by the BAV3 packaging signal. The orientation of the BAV3 fragment is the same as in the BAV3 genome.

pTG 13353: The Ad5 helper plasmid pTG 13353 is similar to pTG 13327 but contains a 0.3 kb encapsidation signal from BAV3 instead of one of 0.8 kb. The final plasmid is produced by homologous recombination between ClaI digested pTG 4656 and PacI-XmaI digested pTG 6462. Transfer plasmid pTG 6462 is similar to pTG 6456 but contains a 0.3 kb ThaI fragment from BAV3 (nt 185-514) instead of the 0.8 kb HaeII-PvuII fragment. The orientation of the BAV3 fragment is the same as in the BAV3 genome.

pTG 13366 & 13367: Similar to the two Ad5 helper constructs containing encapsidation signals from BAV3 of different size (pTG 13327 & 13353), two additional constructs were generated in which the Ad5 encapsidation signal was retained downstream of the BAV3 encapsidation signal. Both constructs were generated like the previous ones by homologous recombination using ClaI linearized pTG 4656 and two transfer plasmids containing the modified 5′ regions. pTG 6460 digested with PacI and XmnI was used for the generation of pTG 13366. pTG 6464, digested with the same enzymes was used to generate pTG 13367. pTG 6460 contains the 0.3 ThaI fragment inserted in the AflIII site at position 151 of the E1 deleted Ad5 genome. The deletion extends from position 459 to 3328 of the Ad5 genome. In order to insert the ThaI fragment, the vector was digested with AflIII and subsequently treated with Klenow. In a similar way, pTG 6464 was generated: instead of inserting a ThaI fragment in the AflIII site, the previously mentioned blunt-ended 0.8 kb HaeII-PvuII fragment was used. In both cases, the orientation of the BAV3 encapsidation signal is the same as in the BAV3 genome.

pTG 14487: Plasmid pTG 14487 contains an E1/E3 deleted backbone from Ad5 flanked by ITRs from BAV3. The BAV3 ITRs are derived from transfer plasmid pTG 14266 which is described before. It contains from 5′ to 3′, the BAV3 5′ ITR, the 0.3 kb bovine packaging signal, the 3′ part of the E1 region from Ad5, a unique HindIII recognition site, part of the Ad5 E4 region and finally the BAV3 3′ ITR. The BAV3 encapsidation signal in this plasmid is replaced by it's homologue from Ad5, which is isolated from pTG 14329. pTG 14329 contains the 5′ ITR from BAV3, the Ad5 packaging signal followed by the 3′ part of the Ad5 E1 region. pTG 14266 is digested with XmnI and DraIII in order to remove the region containing the bovine encapsidation signal, and then recombined with PvuI-MfeI digested pTG 14329 which librates the Ad5 packaging element with large flanking sequences used in the recombination event. The resulting transfer plasmid is named pTG 14485. pTG 14487 is finally generated by homologous recombination between transfer plasmid pTG 14485 digested with HindIII-XhoI and pTG 6401 linearized with PacI. This results in the exchange of Ad5 ITRs in

pTG 6401 by those from BAV3 in pTG 14485.

pTG 14316: described in example 2

pTG 14342: The minimal vector expressing the human Factor IX (hFIX) gene from its endogenous promoter is composed of the 5′ end of Ad5 (nt 1-454), a genomic fragment containing the hFIX gene plus flanking sequences and the Ad5 3′ ITR (nt 35828-35938). The construct is generated by homologous recombination between a transfer plasmid named pTG 14866 (treated with XbaI and calf intestinal phosphatase, CIP) and a plasmid containing a modified version of the hFIX gene (pTG 14869, digested with SfiI). pTG 14866 contains the Ad5 cis-acting elements plus promoter sequences and the 3′ end of the hFIX gene, separated by a unique XbaI site and was constructed as follows. pTG 8343, containing the 5′ end of the Ad5 genome with a deletion in the E1 region is used as a source for the 5′ end of the minimal vector. It is digested with AatII-BglII and overhanging end are made blunt using Klenow enzyme. The Ad5 3′ ITR is isolated from plasmid pTG 5670 which contains the ITR, flanked by a PacI site on one end and a multiple cloning site on the other. The ITR is excised as a BglII fragment, treated with Klenow enzyme and ligated into pTG 8343 prepared as described above. The resulting plasmid, pTG 14138, contains the 5′ end from Ad5 from nt 1 to 454, a MCS and the 3′ ITR from nt 35828-35938. 5′ flanking sequences from the hFIX gene are isolated from pTG 3960 (described in Jallat et al.) as a 1.4 kb fragment by digestion with PstI and HindIII, and introduced in PstI-HindIII digested pTG 14138 by ligation resulting in pTG 14159. Next, 3′ flanking sequences from the hFIX gene are isolated from pTG 3960 as a PCR fragment. To this end, primers OTG 2070 (5′-AGAGCTTGTATGGTTATGGAGG-3′) and OTG 12244 (5′-CACGATACTCGATGCAAGAC-3′) are used to amplify a 1.6 kb fragment which is introduced by ligation in pTG 14159 which was linearized with XbaI, and made blunt using Klenow enzyme. This finally resulted in transfer plasmid pTG 14866. Before hFIX sequences were introduced in this transfer plasmid, we removed 5 consecutive PacI sites present in intron D of the hFIX gene in plasmid pTG 3960. This is necessary, because we will use PacI to excise the final minimal vector from the plasmid vector backbone. If PacI sites are present in the minimal vector sequences, this will result in fragmentation of the minimal vector genome. In order to remove the PacI sites in pTG 3960, the plasmid was digested with PacI, treated with T4 DNA polymerase to remove 3′ overhanging ends and re-circularized. The resulting plasmid is named pTG 14869. In addition to the deletion of the PacI sites, a PvuII fragment is deleted in intron A (4.8 kb) and there is a large deletion in intron F (7.1 kb). These modifications were already present in pTG 3960 and are described in Jallat et al.

pTG 14872: In parallel with pTG 14342, a minimal vector containing hFIX as stuffer DNA was constructed. Instead of using transfer plasmid 14866 containing the 5′ and 3′ flanking sequences of the hFIX gene, pTG 14868 was used. It contains part of intron A downstream of the adenoviral 5′ ITR and encapsidation signal instead of sequences upstream of the hFIX promoter. The construction was started with pTG 14138, which contains the Ad5 cis-acting sequences separated by a MCS (see above) and which was linearized in the MCS by digestion with HindIII followed by treatment with Klenow. Into this vector was ligated a 1.1 kb Scal fragment from pTG 3960 containing part of intron A from the HFIX gene. pTG 14160, with the insert in the sense orientation was thus obtained. Next a unique BamHI site in the remainder of the MCS between the Ad5 packaging signal and the intron A insert was destroyed, while at the same time a NotI site was introduced. This was done by digesting pTG 14160 with BamHI and ligating oligonucleotide OTG 3497 treated with polynucleotide kinase in the BamHI site. OTG 3497 (5′-GATCGCGGCCGC-3′) is capable of forming a NotI site flanked by BamHI compatible cohesive ends by auto-hybridization. The resulting plasmid, pTG 14867, is used to introduce the 3′ flanking region of the hFIX gene needed for homologous recombination in the next step. The 3′ flanking sequence is isolated as an XbaI-KpnI fragment from pTG 14866 and ligated into XbaIKpnI digested pTG 14867. The resulting transfer plasmid pTG 14868 is treated with XbaI and SfiI, which allows for the introduction of hFIX sequences by homologous recombination. The hFIX sequences were isolated from pTG 14869, digested with PvuI and XhoI. Thus a minimal vector construct containing hFIX sequences but lacking the promoter sequences and the first exon of the hFIX gene was obtained. The construct was named pTG 14872.

pTG 14742: We have chosen to introduce expression cassettes in the unique PmeI site of the empty minimal vector pTG 14872. The PmeI site is located in intron D. In order to facilitate introduction of foreign cassettes, we have sub-cloned a fragment containing the PmeI site in a small vector, flanked by NotI sites. Introduction of the expression cassette in the PmeI site can be done in this vector. After excision of the expression cassette with flanking hFIX sequences by the action of NotI endonuclease, the cassette can be introduced by homologous recombination in the empty minimal vector which has been linearized with PmeI. The first step in the construction of the transfer plasmid consisted in introducing an EcoRI site flanked by NotI sites in phagemid pBluescript II SK⁺ which was purchased from Stratagene. The phagemid was digested with PvuII and CIP, thus removing a 445 bp fragment containing the MCS flanked by bacteriophage T3 and T7 promoter sequences. The restriction enzyme recognition sites were introduced by ligation using a polynucleotide kinase treated, self-complementary oligonucleotide (OTG 12753, 5′-CAGGCGGCCGCGAATTCGCGGCCGCCTG-3′). The resulting plasmid is named pTG 15154 and can be used directly to introduce a hFIX fragment. This was done by cutting pTG 15154 with EcoRI and removing terminal 5′ phosphate groups with CIP followed by ligation involving a 3.0 kb MfeI fragment from pTG 3960 containing the PmeI site. The resulting transfer plasmid pTG 14742 can be used directly for the introduction of foreign DNA sequences.

pTG 14899: An example is the introduction of a LacZ expression cassette. The cassette is isolated from the p gal-Control plasmid from Clontech as a BglII-BamHI fragment which is made blunt using Klenow. It is inserted in PmeI-CIP digested pTG 14742 by ligation resulting in transfer plasmid pTG 14900. The cassette was oriented in the same direction as the hFIX gene. After digestion with NotI, the expression cassette containing HFIX flanking sequences was isolated and introduced in PmeI-CIP digested pTG 14872 by homologous recombination. This yielded a minimal vector construct containing a functional LacZ expression cassette (pTG 14899).

pTG 15202: In order to facilitate cloning of various exogenous DNA fragments in the hFIX transfer plasmid, a MCS was cloned in the unique PmeI site of transfer plasmid pTG 14742. The MCS was isolated as a NotI fragment from pPoly III-I which was made blunt by the action of Klenow. The resulting plasmid pTG 15179 has a large number of unique sites in the former PmeI site which will facilitate subsequent cloning steps. The LacZ expression cassette for the new minimal vector was isolated as a SalI-BglII fragment from plasmid pTG 15103. Briefly, the fragment contains the RSV promoter followed by intron 2 from the rabbit β1 globin gene, the LacZ gene with the SV40 nuclear localization signal fused to at the N-terminus and finally the SV40 polyadenylation signal. This fragment was ligated into SalI-BglII digested pTG 15179 yielding transfer plasmid pTG 15199. This transfer plasmid was digested as usual with NotI to liberate the expression cassette flanked by hFIX sequences, and this fragment was introduced in the empty minimal vector pTG 14872 by homologous recombination after digestion of the latter with PmeI-CIP. The new minimal vector containing the LacZ gene under expression of the RSV promoter (pTG 15202) has higher b-galactosidase activity than minimal vector pTG 14899.

pTG 14927 & 14929: Two chimeric Ad5 helper genomes containing a functional E1 region derived from Ad5 and two encapsidation signals each were constructed in parallel. Plasmids pTG 6460 & 6464 were used as precursors for the construction and were described above. They contain the bovine encapsidation signal in the form of the 0.3 kb ThaI fragment (pTG 6460) or the 0.8 kb HaeII-PvuII fragment (pTG 6464) inserted in the AflIII site between the Ad5 5′ ITR and the Ad5 encapsidation signal. Downstream of these elements is part of the Ad5 E1 region and the E2B region. The missing part of the E1 region is re-introduced between the Ad5 encapsidation signal and the downstream part of the E1B region in both plasmids by homologous recombination. Both plasmids are digested with BglII and SalI, both of which cut between the Ad5 encapsidation signal and the downstream part of the E1B region. The missing E1 sequences are isolated from pTG 3602 (wild-type Ad5) as a 5.8 kb EcoRI-XhoI fragment. After the recombination reaction, the following plasmids were obtained: pTG 14926, derived from pTG 6460 and pTG 14928, derived from pTG 6464. Both of the newly created plasmids were then used to reconstitute the entire chimeric genome, again by recombination. Both plasmids were digested with PacI and XmnI, which cut just upstream of the 5′ ITR and in the plasmid backbone. Recombination is carried out with pTG 4656 digested with ClaI. This results in wild-type E1/E3° adenoviral vectors which have an extra 0.3 kb (pTG 14927) or 0.8 kb (pTG 14929) bovine packaging signal.

pTG 15356-15369 & 15155: A series of 4 different chimeric vectors was constructed with the following common background: BAV3 5′ ITR, Ad5 E1 region driven by the murine PGK promoter, Ad5 E3° backbone and a BAV3 3′ ITR. The constructs differ with respect to two sequence elements from BAV3 that will be tested for their involvement in packaging. The first is the 0,8 kb HaeII-PvuII fragment from the 5′ part of the BAV3 genome which is inserted between the 5′ bovine ITR and PGK promoter in pTG 15356 & 15357. The second a 120 bp sequence from the 3′ part of the BAV3 genome, between the SspI site and the Apol site, which is also present in pTG 15356 and in pTG 15358. pTG 15369 lacks both elements. Additionally a control construct was made which is similar to pTG 15356 but that lacks the Ad5 E1 region and the PGK promoter.

pTG 15356: The construction of Ad5 helper construct pTG 15356 was started with transfer plasmid pTG 14328, which has been described above. Apart from internal sequences derived from the Ad5 genome it contains the 5′ and 3′ ITRs from BAV3 but lacks any encapsidation region. First of all, the 3′ bovine sequence (ITR) in this plasmid is enlarged by adding the abovementioned 120 bp fragment. This fragment, including the BAV3 3′ ITR is isolated from pTG 5451 by digestion with BglII and SspI. pTG 14328 is digested with SalI which cuts at the junction between the 3′ ITR and the Ad5 sequences and made blunt with Klenow enzyme. The two plasmids are joined by ligation and recombination. Ligation takes place between the blunted SalI site in pTG 14328 and the DNA end generated by digestion with SspI which is already blunt. Recombination then takes place at the level of the 3′ ITR resulting in pTG 15141. The net change is enlargement of the 3′ sequences derived from BAV3. Next, the enlarged 3′ sequence is excised using PacI and HindIII and introduced in pTG 14315 by homologous recombination. pTG 14315 is a transfer plasmid that already contains the 0.8 kb BAV3 packaging element in the 5′ region. To introduce the enlarged 3′ ITR, pTG 14315 was cut with BamHI and SalI, both of which cut at the junction between the Ad5 E4 region and the BAV3 ITR. The transfer plasmid thus obtained (pTG 15143) contains two “enlarged” BAV3 ITRs. Next, a functional E1 region under control of the mPGK promoter was introduced in the transfer plasmid. The PGK-E1 cassette was derived from expression plasmid pTG 4671 which has been described in Imler et al., 1996, Gene Ther. 3 75-84. The PGK-E1 cassette is almost entirely present in a 4 kb SalI-MfeI fragment. Only the last part of the pIX gene and the polyadenylation signal are missing. It is this fragment that was used in a ligation-recombination reaction with pTG 6456 which was linearized with SalI and treated with CIP. pTG 6456 contains the 5′ end of transfer plasmid pTG 15143 including the end of the Ad5 E1 region and the beginning of the E2B region. The SalI site is situated between the 0.8 kb BAV3 encapsidation region and the downstream part of the E1B region. The ligation took place between the end of the BAV3 packaging signal from pTG 6456 and the PGK promoter of the E1 expression cassette. Subsequent recombination between sequences at the end of the E1 region reconstituted a circular plasmid (pTG 15126) with all complete E1 open reading frames preceded by a PGK promoter. We have used this plasmid to introduce the same sequences in pTG 15143. To this end, pTG 15143 was digested with AvrII in order to linearize the plasmid at the level of the 0.8 kb packaging signal and to allow for homologous recombination with a fragment containing the PGK promoter and the missing E1 sequences. Unfortunately there is a second AvrII site present in the E4 region included in pTG 15143. We therefore digested the plasmid only partially, and carried out the recombination with a 5.0 kb PacI-MfeI fragment from pTG 15126, which contains the Ad5 ITR, the bovine 0.8 kb encapsidation signal, the murine PGK promoter followed by the AdS E1 region up to the MfeI site in the E1B region. The resulting transfer plasmid, pTG 15294, has the PGK-E1 region inserted and bovine ITRs. It can be used for the final step: reconstitution of chimeric genome pTG 15356. This was done by homologous recombination between pTG 3652, from which an E3 deleted Ad5 genome was liberated by digestion with PacI, and pTG 15294 digested with PsiI. PsiI cuts the transfer plasmid in the E1B region and the E4 region, leaving enough flanking sequences for homologous recombination to take place.

pTG 15357: The second Ad5 helper construct does not contain the 120 bp sequence derived from the 3′ region of BAV3. The construction was therefore started with the introduction of the PGK promoter and the Ad5 E1 region in transfer plasmid pTG 14315 which contains the 0.8 kb bovine encapsidation signal. The 5.0 kb PacI-MfeI fragment from pTG 15126 was used again and recombined with XmaIII-AflIII digested pTG 14315. These enzymes cut just before and after the junction between the packaging signal and the 3′ end of the E1B region. The resulting transfer plasmid pTG 15295 was used to construct the final chimeric vector by homologous recombination. Like in the construction of the previous helper vector, pTG 15295 was digested with Psil and recombined with PacI digested pTG 3652, yielding pTG 15357

pTG 15358: The third Ad5 helper construct retains the 120 bp sequence but lacks the 0.8 kb bovine encapsidation signal. An intermediary plasmid was constructed based on pTG 15126 which lacks the 0.8 kb bovine encapsidation signal. This was achieved by replacing a PacI-SalI fragment containing the Ad5 ITR and bovine encapsidation element in pTG 15126 by a PacI-SalI fragment from pTG 14261 containing the BAV3 ITR. The resulting plasmid pTG 15304 was used to insert PGK-E1 sequences in transfer plasmid pTG 15141 in the following way: pTG 15141 was digested with DraIII which cuts twice in what is left of the E1B region and recombined with a 4.3 kb PacI-MfeI fragment from pTG 15304. This results in transfer plasmid pTG 15320 which, after digestion with PsiI and recombination with PacI digested pTG 3652 gives the final Ad5 helper construct pTG 15358.

pTG 15359: The last E1 expressing Ad5 helper construct serves as a control and contains none of the two bovine sequences are being tested for their involvement in encapsidation. It is made in an analogous way as pTG 15358 using E1 expression plasmid pTG 15304 and transfer plasmid 14328. The former is digested with PacI and MfeI and the latter with Drall, and the two are recombined producing transfer plasmid pTG 15316. This plasmid is digested with Psil and recombined with PacI digested pTG 3652 yielding pTG 15359.

pTG 15155: The E1° counterpart of Ad5 helper construct pTG 15356 was made using an intermediary plasmid in the construction of pTG 15356. pTG 15143 is a transfer plasmid that is deleted for Ad5 E1 but contains both 5 and 3′ sequence elements in addition to the BAV ITRs. Residual E1B-E2B and E4 sequences allow for the introduction by recombination of the remainder of the Ad5 genome.

The helper construct pTG 15155 is finally obtained as follows: pTG 15143 is digested with Hind III and XhoI and recombined with PacI digested pTG 14316.

2. Cell Culture Conditions

Cells are cultured in DMEM medium supplemented with 10% fetal calf serum, 2 mM L glutamine, gentamycin and 0.1 mM minimal amino acids. Cells are incubated at 37° C. in a humidified atmosphere containing 5% CO2.

3. Transfections

Producer cells are transfected with Pac I digested plasmids containing recombinant adenoviral genomes using the standard calcium phosphate transfection technique. For 10 cm dishes 2×10⁶ cells are transfected with a maximum of 30 μg plasmid DNA, which is diluted in 0.05× TE (total volume 420 μl). 60 μl 2 M CaCl2 is added to the DNA and the mixture is slowly added to 480 μl 2× HBS while vortexing. After incubation at room temperature for 20-30 minutes the precipitate is added to the cells. The culture medium of the cells was previously replaced by 10 ml fresh complete medium. Cells are incubated overnight and washed the next day with complete medium. Finally, cells are incubated in the presence of 10 ml medium containing 10% fetal calf serum at 37° C./5% CO2. For transfection of cells in 6-well-plates (6wp) a maximum of 6 μg plasmid DNA is used. The number of cells per well is 5×10⁵. For the rest the procedure is the same as for 10 cm dishes.

Alternatively, transfections were carried out using SuperFect or PolyFect from Qiagen according to the manufacturers protocol.

4. Transduction of Target Cells

Typically, target cells are transduced as follows: cells have been plated the previous day in a 6 well plate at a density of 5×10⁵ cells per well. Medium is replaced by 2 ml DMEM with 2% fetal calf serum and 200 μl virus containing supernatant or indicated amounts of purified virus. Cells are incubated overnight at 37° C./5% CO2 in a humidified atmosphere. Then the culture supernatant is replaced by 2.5 ml complete DMEM and incubation is continued until cells are analysed.

B. Results

1. Design of the Basic Set of Ad5 Helper Constructs

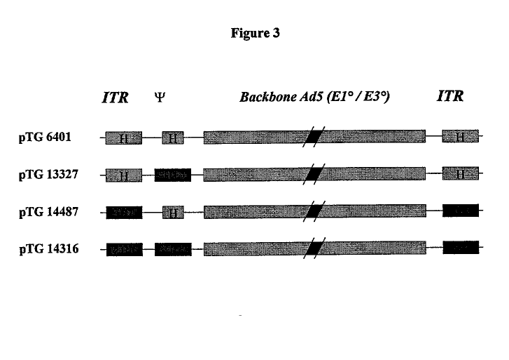

Four different Ad5 helper constructs have been designed (FIG. 3). Each of these constructs will be tested in various bovine cells in the presence and absence of BAV3 helper virus, to see if they can be efficiently produced. These constructs will also be tested in a number of human cells and the production of minimal viruses will be determined.

pTG 6401 is described in Example 2. It is an E1/E3 deleted type 5 adenovirus which serves as a positive control. pTG 13327 is derived from pTG 6401 and has a bovine encapsidation region instead of the encapsidation region from Ad5. Due to the presence of Ad5 ITRs it is expected to replicate it's DNA in human cells complementing for E1 functions. Due to the presence of the bovine encapsidation region it could be packageable in bovine cells in the presence of a BAV helper virus, but not in human cells. pTG 14316 is also derived from pTG 6401 and has the bovine ITRs in addition to the bovine encapsidation region. At the 5′ end the bovine ITR and encapsidation region are contiguous. This construct should also be packaged in bovine cells in the presence of BAV3 helper virus. pTG 14487 is a negative control construct from which the endogenous ITRs are replaced by BAV3 ITRs (encapsidation region is from Ad5).

2. DNA Replication of E1-deleted Ad5 Helper Constructs in Human Cells

The synthesis of adenoviral DNA of the chimeric Ad5 helpers was tested by analyzing its sensitivity towards digestion with Dpn I & II. After transfection of the DNA in 293 cells, low molecular weight DNA was isolated after 2, 4, 6 & 10 days. This DNA was digested with Dpn I and II. These enzymes both recognize the restriction site GATC, but have different sensitivities towards methylation of the A residue. When the A is methylated, as is the case in the plasmid DNA, Dpn I cuts, but Dpn II does not. When the A is not methylated, as is the case after replication in eukaryotic cells, the situation is reversed.

After transfection of the pTG6401, pTG13327, pTG14487 and pTG14316 constructs in 293 cells, only cells transfected with pTG 6401 showed cytopathic effect (CPE) starting after 4 days, followed by complete detachment of the cells after one week. All other cultures, even after incubation times exceeding 2 weeks, did not show signs of CPE.

The results of the DNA synthesis assay clearly show that only pTG 6401 and pTG 13327 are able to amplify their DNA in 293 cells. Both constructs contain the Ad5 ITRs. The constructs that contain the BAV3 ITRs did not amplify, indicating that the BAV3 ITR does not function in human cells in the presence of Ad5 E2 proteins. Since pTG 13327 is able to synthesize it's DNA but does not show CPE, it is likely that the defect is attributable to a packaging defect.

3. Detection of Minimal Vector Production using Southern Blots

The Ad5 helper constructs were tested for their ability to amplify MV DNA and to package this DNA in infectious viral particles. The vector pTG 8595 that has the E1 and E4 regions deleted was included in this study in addition to the basic set of helper constructs. This virus is not able to synthesize DNA efficiently. DNA was introduced using the calcium phosphate transfection technique into 293 (Graham et al., 1977, J. Gen. Virol. 36, 59-72) and PER.C6 (Fallaux et al., 1998, Human Gene Ther. 9, 1909-1917) cells. Both Ad5 helper constructs and a minimal vector expressing LacZ (pTG 14899) were transfected (3 wells/construct). Two, five and eight days after transfection, DNA was isolated and analyzed by the Dpn I/II assay. Virus, eventually produced 8 days after transfection was passed on fresh 293 or PER.C6 cells. Hirt DNA was isolated from all cultures and the presence of Ad5 helper constructs and MV was analyzed by southern blot using an Ad5 IVa2 probe as well as a LacZ probe.

Identical results were obtained with 293 and PER.C6 cells. DNA synthesis is seen for Ad5 helper constructs pTG 6401 and pTG 13327 confirming previous results. No DNA synthesis was detected for pTG 8595, 14316 or 14487. Furthermore, all helper constructs are able to synthesize MV DNA, confirming that all viral proteins involved in DNA synthesis from each helper construct function correctly. No minimal vector DNA was seen after the first passage, whatever the AdS helper construct used. It is likely that formation of MV is very inefficient, at least in the first passage, and thus escapes detection.

4. Detection of Minimal Vector Production using the LacZ Reporter Gene

A new MV construct has been used in which the LacZ gene is placed under the control of an RSV promoter (pTG 15202), which is expected to be more efficient than the SV40 promoter present inpTG 14899.

The same AdS helpers were used as before to complement the LacZexpressing MV construct pTG 15202. PER.C6 cells were transfected and 1 week later, cells were harvested. After freeze/thawing and centrifugation, supernatants were used to infect fresh PER.C6 cells. One day later, cells were fixed and stained for beta-galactosidase activity. As a result, all AdS helper constructs were able to produce infectious MV with comparable efficiency.

It is known from the literature that expression of late viral proteins depends on the replication of the adenoviral genome (Thomas and Mathews, 1980, Cell 22, 523-533). These results indicate that this is not the case with our constructs. Without late viral protein synthesis, it is inconceivable that infectious viral particles can be produced. Our observations can be explained in two ways. Firstly, it is possible that the technique used to detect viral DNA replication is not sensitive enough, and that some DNA replication has occurred with the constructs containing bovine ITRs. Secondly, DNA replication did not occur, and consequently, late viral protein synthesis does not depend on template replication in our system. Both situations are advantageous: in both cases, it is possible to use chimeric helper constructs that have bovine ITRs. The constructs derived from pTG14316 are therefore very interesting candidates for our strategy. In addition, if DNA replication turns out not to be necessary for the production of late viral proteins, it is conceivable to provide helper functions for the production of minimal viruses in a form that does not depend on active replication, such as episomes.

5. Coinfection of MDBK and A549 Cells with BAV3 and Ad5 Helper Viruses

We investigated whether it is possible to find conditions under which BAV3 helper can amplify Ad5 helper constructs in bovine cells. Since bovine cells are difficult to efficiently transfect, we have tried to find conditions in which BAV3 viruses can co-exist with Ad5 viruses in bovine cells. We have used noncomplementing bovine (MDBK) and human cells (A549). Different MOIs for the viruses were used. Single infections were performed with a wild-type Ad5 (Ad5WT), a E1°/E3° AdS, a BAV3 wild-type (BAV3WT) or a E1A° BAV3 virus. Co-infections were also performed with Ad5 and BAV3 viruses, respectively Ad5WT and BAV3WT, Ad5WT and E1A°BAV3, E1°/E3° Ad5 and BAV3WT and E1°/E3°Ad5 and E1A°BAV3. The DNA was recovered from A549 and MDBK cells and analyzed by Southern blot after HindlIl digestion using Ad5 and a BAV3 probes.

The single infections show that only WT Ad5 is able to grow in A549 cells whereas both WT BAV3 and WTAd5 viruses are able to grow in bovine cells.

When MDBK cells are infected with both WT BAV3 and WT Ad5 viruses together they can be co-amplified. It should however be noted that WT BAV3 tends to outgrow Ad5 when more passages are performed. When MDBK cells are infected with WT Ad5 and BAV3 E1A° viruses we observe efficient mobilization of BAV3 E1A° by Ad5. BAV3 E1A° is replicating more efficiently than WT Ad5, even when Ad5 WT is initially present in excess, but Ad5 remains present. The reverse situation, rescue of Ad5 E1° by BAV3 is not observed, presumably due to the large growth advantage of BAV3.

All virus combinations have also been used to infect human A549 cells. The only virus that grows is WTAd5, even when BAV3 viruses are initially present in 10 fold excess.

This result shows that both viruses can be co-amplified, and moreover shows that E1 complementation by Ad5 can be used to control the growth of BAV3 virus in bovine cells. This is an important finding since it shows that the simultaneous expression of bovine and human viral proteins does not prevent the formation of infectious virus. One way to ensure that Ad5 viruses will co-exist with BAV3 helper, is to make expression of BAV3 dependent on the presence of Ad5 helper in the same cell.

6. Production of pTG 13327 Derived Ad5 Helper Virus in Bovine Cells

Production of new Ad5 Helper Constructs in Human Cells

The BAV3 helper virus is adapted to growth in bovine cells, and will thus have a selective advantage over Ad5 helper viruses, especially when the latter are modified with BAV3 sequences. To avoid or minimize the outgrowth of BAV3 helper over Ad5 helper during the co-amplification process in bovine cells, it is possible to make replication of BAV3 dependent on Ad5. A first alternative is to use E1 deleted BAV3 together with E1 expressing Ad5 in non-complementing bovine cells. In this case, replication of BAV depends directly on the expression of the E1 region from Ad5. We have shown that in this case, both viruses can co-exist for at least three passages, the maximum number of passages tested.

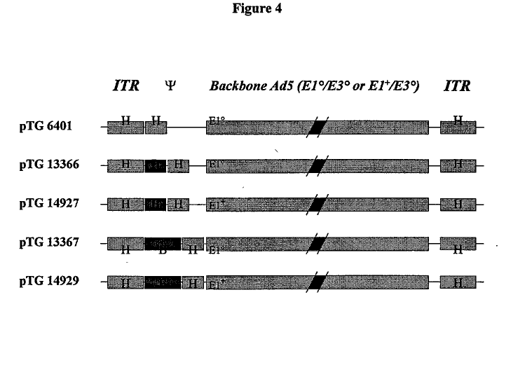

A number of new constructs which exploit complementation of BAV3 in bovine cells by Ad5 helper are illustrated in FIG. 4. For this purpose, the Ad5 E1 region was reintroduced in pTG 13327 (and pTG 13353, which contains a smaller BAV3 encapsidation region). Since the promoter elements of the E1 region are located in the encapsidation region, we also re-introduced the Ad5 encapsidation region. The resulting constructs pTG 14927 and 14929 thus have BAV3 and Ad5 encapsidation regions. Due to the presence of the Ad5 encapsidation region it will be possible to produce these viruses in human cells. The new constructs were transfected in 293 cells as well as two E1° control constructs (pTG 13366 and 13367) or the pTG 6401 control.

The introduction of a sequence element between the ITR and the encapsidation region of Ad5 may result in delayed growth characteristics with respect to the wild type virus (Hearing et al., 1987, J. Virol 61, 2555-2558). This was again observed when the constructs indicated in FIG. 4 were transfected in 293 cells. pTG 6401 transfection yields plaques after 4-5 days whereas pTG 13366 and 14927 need a week and pTG 13367 and 14929 approximately 10 days before plaques begin to form. When the viruses are passed this effect is seen again, both on 293 cells and A549 cells. When tested on bovine cells, the effect is even more pronounced. Both on MDBK cells and VIDO-R2 cells, pTG 14927 and 14929 have an attenuated phenotype.

The adenoviral DNA isolated from different cell lines (293, A549, VIDO-R2 and MDBK) was analyzed by Southern blot after HindlIl digestion and probed with an Ad5 probe specific for the Iva2 gene or a BAV3 probe specific for the encapsidation region. As a result, all viruses can be propagated on 293 cells and VIDO-R2 cells, both of which complement for Ad5 E1 functions. Only pTG 14927 and 14929 can be propagated on A549 cells and MDBK cells, which shows that they express the E1 region. Southern analysis with the BAV3 probe indicates that all chimeric viruses indeed contain a bovine encapsidation region.

Co-infections of A549 and MDBK cells

AdTG 14927 and 14929 viruses were produced in A549 cells in larger quantities and were tested in human and bovine cells together with BAV3 helper viruses. The BAV3 viruses that were used are the WT virus and the E1 deleted virus (BAV3 E1A). The adenoviral DNA isolated from co-infected A549 cells was analysed by Southern blot after HindlIl digestion and probed with the IVa2specific Ad5 probe or the psi-specific BAV3 probe. As a result, the only viruses that can grow are Ad5 WT and the chimeric viruses AdTG 14927 and 14929. No bovine viruses were detected. As detected with the BAV3-specific probe, the chimeric viruses contain the bovine psi element. In the case of co-infections with Ad5 WT and either BAV3 WT or BAV3 E1A, both viruses can co-exist for at least 3 passages confirming the results of the previous experiment. In both cases however, the BAV viruses are becoming more prominent. In the case of coinfections using the chimeric viruses, the BAV viruses grow very well, but the chimeric viruses are lost very rapidly. It is possible that the chimeric viruses were lost due to recombination events with the BAV3 helper viruses in the encapsidation region and perhaps other regions in the viruses. However, no recombination events could be detected using southern blots.

The presence of the Ad5 helper viruses was also tested in passages 2 and 3 of MDBK cultures by passing supernatants of these cultures on fresh A549 cells. In all cases Ad5 WT and the two chimeric viruses were amplified without any apparent modification of the viral genomes. This proves that the chimeric virus was present during all the passages, but at very low levels that could not be detected using southern blots.

In summary, moving the Ad5 encapsidation region away from the 5′ end of the genome has a negative impact on the growth properties of the virus. The further away, the slower the virus grows. This is in agreement with the literature but we find that the Ad5 encapsidation region can be moved farther away than 600 bp as is stated in the literature.

pTG 13327 derived AdS helper vectors can not be amplified in the presence of E1 A deleted BAV3 helper virus, whereas Ad5 WT virus can. The only difference (apart from the presence of the E3 region in WT AdS) is the insertion of a bovine sequence between the 5′ ITR and the Ad5 encapsidation region in Ad5 helper. Recombination might have occurred between these bovine sequences in the chimeric helper viruses and the BAV3 helper construct. This leads to viruses with two different ITRs, which is probably deleterious for the replication of their genomes. If recombination is not complete, a low level of unaltered Ad5 helper can persist, explaining E1 complementation of BAV E1A° in subsequent passages.

It will be interesting to see if AdS helper constructs derived from pTG 14316 which contains the first contiguous 1 kb of BAV3 sequences will be better helper candidates. Such constructs will eliminate the problem of recombination with BAV3 helper, since recombination at the level of the bovine sequences in both viruses will have no net effect.

7. Production of pTG 14316 derived Ad5 Helper Virus in Bovine Cells

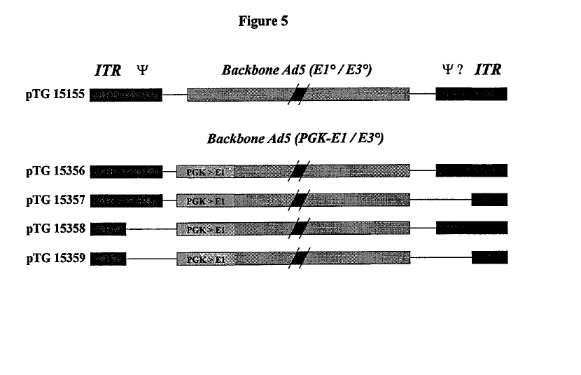

The design of AdS helper constructs derived from pTG 14316 is outlined in FIG. 5. pTG 15356-59 differ with respect to two BAV3 sequences that will be tested for their implication in packaging. All constructs express Ad5 E1 from a murine PGK promoter. The original AdS E1 promoter sequences were replaced by the PGK promoter to avoid that overlapping encapsidation elements from the Ad5 E1 promoter are present in the helper constructs. pTG 15155 is an E1° control for pTG 15356.

Cross-complementation between BAV3 and Ad5 helper viruses can be accomplished in non-E1 complementing bovine cells such as MDBK cells. The VIDO-R2 cells have also been used for the first step but these bovine cells express the AdS E1 region, so that cross complementation will not work. All helper constructs have been transfected in 10 VIDO-R2 cells in 6-well plates using the calcium phosphate precipitation technique. After 1 day individual cultures were infected with BAV3 E1A° virus at an MOI of 0.1. After another two to three days, all super-infected cells had lysed and cells were harvested.

Subsequently, after three freeze/thaw cycles of the VIDO-R2 cells, MDBK cells were infected using half of the material produced. After 1 week, the cultures containing AdTG 15356 or 15357 (plus BAV3 E1A°) started to show CPE and were completely lysed 3 days later. After one more passage on MDBK cells, lysis was readily observed for AdTG 15356 and 15357. No lysis occurred with constructs pTG 15358, 15359 or pTG 15155.

Viral DNA was isolated from the cultures, digested with HindII and analyzed using southern blotting probed with the Ad5 or BAV3-specific probe. As a result, all Ad5 helper constructs that maintain the 0.84 kb element from BAV3 at the 5′ end, can be propagated from one culture to the next. The presence of the 120 bp 3′ element does not seem to play a role.

In contrast to pTG 13327 derived helper vectors, pTG 14316 derived constructs can be amplified in bovine cells in the presence of BAV3 E1 A° using E1 based cross-complementation. This supports the hypothesis that recombination in the bovine encapsidation region at the 5′ end of Ad5 and BAV3 helper constructs was the cause of the rapid decrease of pTG 13327 derived vectors.

These results also show that the 0.84 kb BAV3 fragment is essential for the encapsidation of Ad5 helper constructs in our experimental setup. This is in fact the first experimental proof that the 0.84 kb sequence is indeed involved in packaging.

8. Minimal Vector Constructs and Transfer Plasmids

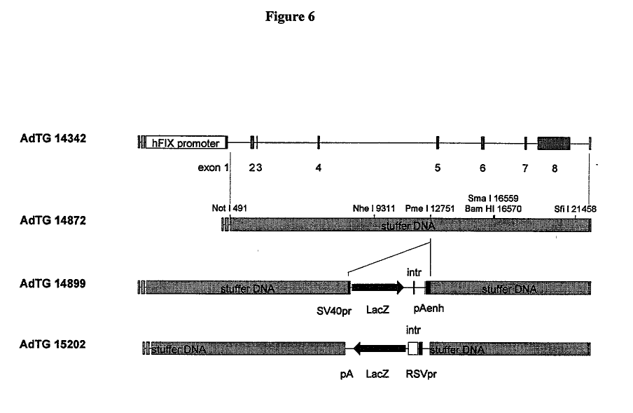

Two minimal constructs were constructed using the hFIX gene as illustrated in FIG. 6. pTG 14342 is able to express the hFIX gene from its own endogenous promoter, whereas pTG14872 contains hFIX sequences as stuffer DNA. The total size of the last construct is 22.4 kb, which means that the size of an expression cassette to be inserted should be comprised between 4,5 and 15 kb. pTG 14872 is the “empty minimal vector” in which expression cassettes can be introduced. The first expression cassettes that were inserted was the reporter gene LacZ driven by the SV40 promoter (pTG 14899) or the RSV promoter (pTG 15202) using the unique Pme I restriction enzyme recognition site (position 12751 in pTG 14872) to introduce the expression cassettes. The first step in the cloning procedure was the sub-cloning of a fragment comprising the Pme I site plus flanking sequences of approximately 1 kb resulting in transfer plasmid pTG 14742. The sub-cloned fragment is flanked by two Not I sites. The expression cassettes were cloned directly in the blunt Pme I site. To introduce the expression cassette in the empty MV backbone, the expression cassette plus flanking HFIX sequences was excised from the transfer plasmid by digestion with Not I and introduced in Pme I digested empty MV by homologous recombination.

In order to facilitate future cloning strategies, a multiple cloning site (MCS) can be introduced in the Pme I site of transfer plasmid pTG 14742 resulting in pTG 15179. Due to the presence of a larger number of unique restriction enzyme recognition sites, cloning of expression cassettes is now much easier. A similar transfer plasmid has been constructed that can be used to introduce DNA fragments at position 16560 in pTG 14872. This region of the empty MV corresponds to a multiple cloning site in a deleted region in intron F of the HFIX stuffer sequence. The multiple cloning site is a result of the cloning procedure used to make the hFIX expression plasmid (pTG 3960, Jallat et al, EMBO J. 9, 3295-3301). Two changes were made. First, a hFIX fragment containing the MCS was sub-cloned in intron F. In the resulting transfer plasmid pTG 15299 the MCS containing fragment is flanked by Sfi I and Pac I sites allowing for more flexibility to excise the insert from the transfer plasmid. As with the previously described transfer plasmid, this new transfer plasmid can be used to quickly introduce expression cassettes. Second, the MCS was replaced in pTG 15299 by an Asc I site, and used the resulting plasmid pTG 15338 to introduce the Asc I site in the empty MV backbone pTG 14872, resulting in pTG 15384

Transfer plasmids pTG 15179 and 15299 can be used to introduce two independent DNA fragments in the new empty MV vector pTG 15384. Both transfer plasmids have an MCS and the inserts are flanked by recognition sites for different rare cutting restriction enzymes. To introduce foreign DNA fragments present in the transfer plasmids into the empty minimal vector, the empty vector can be linearized with either Pme I or Asc I.

To further optimize the empty MV, a 4,8 kb fragment from FIX intron A that is deleted in pTG 14872 can be re-introduced. This would bring the size of the empty MV to 27,2 kb. This is exactly the minimal size required for efficient packaging. The resulting vector thus allows for the insertion of small foreign DNA fragments, something that is not possible with the current one. In addition, it would be advantageous to remove some repetitive sequences and to introduce Swa I sites next to the Pac I sites flanking the ITRs which can be used to linearize the minimal vector prior to transfection in producer cells.

9. Production of Ad5 Helper Virus using Non-bovine Cell Lines

The experimental data discussed above show that amplification of Ad5 helper in bovine cells can be achieved. Propagation of the Ad5 helper virus depends on packaging functions provided by the BAV3 virus. One way to augment production of Ad5 helper, can be to change the cell line that produces Ad5 helper. Switching production from bovine to non-bovine cells might compromise the growth advantage of BAV helpers. Such a cell line should be capable of propagating BAV3. Preliminary experiments indicate that cotton rat lung fibroblasts can indeed produce Ad5 helper in the presence of BAV3 helper and that the ratio between Ad5 and BAV3 helper is more favorable than with MDBK. The use of non-bovine cells has the additional advantage that they are more acceptable for clinical experiments, since they are not related to transmittable diseases such as bovine spongiform encephalopathy.

10. New Cross-complementation Strategy Based on the Ad E2 Region

To prevent or minimize rapid outgrowth of the the BAV3 virus over the Ad5 helper constructs, it will be advantageous to employ a strategy based on complementation in which replication of the BAV3 virus is made dependent on the presence of Ad5 helper constructs in the same cell. Example 4.7 illustrates E1 complementation. However cross-complementation strategy based on additional regions can also be envisaged. It is possible to delete an essential structural gene from BAV3 and complement it by it's homologue from Ad5. A screening can be performed, knocking out one by one all BAV3 structural proteins, and checking if one or more particular Ad5 genes can functionally complement missing BAV3 genes.

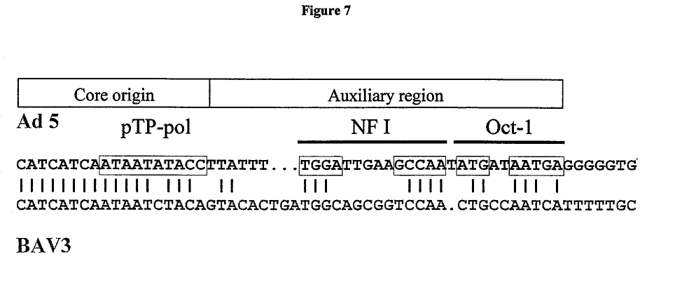

An alternative can be proposed based on the genes involved in the replication of adenoviral DNA. The three viral E2 genes involved, pol, pTP and DBP, function in DNA synthesis. They interact with sequences in the penultimate basepairs of the adenoviral genome, which constitutes the origin of replication (ori, FIG. 7). Sequence differences in the BAV3 and Ad5 ori's can be exploited for complementation. In the pTG 14316 derived Ad5 helpers, the ITRs and therefore the ori are from BAV3. Co-infections of human cells (239, PER.C6 or A549) with Ad5 and BAV3 viruses show that the BAV3 ITR is not recognised by the Ad5 E2 proteins since the BAV3 genome is not amplified in the presence of Ad5 viruses. If the ITR of BAV3 is replaced by the ITR from Ad5 in both Ad5 helper and BAV3 helper constructs, replication of BAV3 DNA will depend on the presence of Ad5 E2 proteins. Subsequently, the BAV E2 homologues can be deleted from the BAV3 helper virus, thus rendering this construct strictly dependent on Ad5 helper for its replication.

The conversion from BAV ITR to Ad5 ITR can be carried out in several steps. Change of the penultimate 20 basepairs containing the core origin, the penultimate 50 basepairs containing the entire ori, or the entire ITR.

1. A method for preparing a recombinant minimal adenoviral vector stock comprising:
(a) Introducing in a first cell line (i) a first helper adenoviral vector or virus and
(ii) a second helper adenoviral vector or virus the genome of (i) and (ii) comprising 5′ and 3′ ITRs, a encapsidation region and one or more gene(s) of the early and late regions,
The genome of (i) deriving from a first adenovirus genome,
The genome of (ii) deriving from a second adenovirus genome different from said first adenovirus with the exception of at least the encapsidation region which derives from said first adenovirus genome,
Said first helper (i) being capable of packaging said second helper (ii) in said first cell line;
(b) culturing the cell obtained in step (a) under appropriate conditions to allow the production of viral particles comprising (ii) and, optionnally (i),
(c) recovering the viral particles obtained in step (b) from the cell culture,
(d) introducing in a second cell line said viral particles obtained in step (c) and a recombinant minimal vector,
(e) culturing the cell obtained in step (d) under appropriate conditions to allow the production of viral particles comprising said recombinant minimal vector, and
(f) recovering the viral particles obtained in step (e) from the cell culture.

2. The method of claim 1, wherein said first adenovirus is an animal adenovirus and said second adenovirus is a human adenovirus.

3. The method of claim 2, wherein said first adenovirus is a bovine adenovirus and said second adenovirus is a human adenovirus.

4. The method of claim 3, wherein said first adenovirus is BAV3 and said second adenovirus is Ad5.

5. The method of any preceeding claims, wherein said first and/or second helper adenoviral vector is (are) a wild-type adenovirus genome(s).

6. The method of any preceeding claims, wherein said first and/or second helper adenoviral vector is (are) a defective mutant(s) of a wild-type adenovirus genome.

7. The method of claim 6, wherein said first and second helper adenoviral vectors are defective mutants of wild-type adenovirus genomes and are capable of cross-complementing each other for at least one defective function.

8. The method of claim 6 or 7, wherein said first helper adenoviral vector is defective for E1 function.

9. The method of any of claims 6 to 8, wherein said first helper adenoviral vector is defective in E2 function.

10. The method of claim 9, wherein said defective E2 function is caused by a mutation or deletion in at least the gene encoding DBP, Pol and/or pTP.

11. The method of any of claims 6 to 10, wherein said second helper adenoviral vector is defective for E1 function and optionally E3 function.

12. The method of claim 11, wherein said second adenoviral heper vector is an AdS genome deleted of nucleotides approximately 455 to approximately 3327 and having nucleotides approximately 149 to approximately 454 comprising the Ad5 encapsidation region replaced by nucleotides approximately 141 to approximately 984 of the BAV3 genome.

13. The method of any preceeding claims, wherein said second adenoviral vector is functional for the E1 function and wherein the E1 region is placed under the control of a non-adenoviral promoter

14. The method of any preceeding claims, wherein said first and second adenoviral helper vectors have an origin of replication recognized by the same E2-encoded gene products.

15. The method of claim 14, wherein the endogenous 5′ and 3′ ITRs of the first adenoviral helper vector are modified to make the origin of replication recognized by the E2 gene products expressed from the second adenoviral helper vector.

16. The method of claim 15, wherein said modification consists in the replacement of:
the penultimate 20 bp containing the core origin,
the penultimate 50 bp containing the entire origin of replication or
the entire ITRs of said first adenoviral helper vector by the equivalent sequence of the 5′ and 3′ ITRs of said second adenoviral helper vector.

17. The method of claim 14, wherein the endogenous 5′ and 3′ ITRs of the second adenoviral helper vector are modified to make the origin of replication recognized by the E2 gene products expressed from the first adenoviral helper vector.

18. The method of claim 17, wherein the endogenous 5′ and 3′ ITRs of said second helper adenoviral vector are replaced by the 5′ and 3′ ITRs of said first adenovirus genome.

19. The method of claim 18, wherein said second helper adenoviral vector is an Ad5 genome deleted of nucleotides approximately 455 to approximately 3327 and nucleotides approximately 28592 to approximately 30470 and having nucleotides approximately 1 to approximately 454 comprising the ITR 5′ and the Ad5 encapsidation region replaced by nucleotides approximately 1 to approximately 984 of the BAV3 genome and nucleotides approximately 35826 to approximately 35935 comprising the ITR 3′ replaced by nucleotides approximately 34188 to approximately 34446 of the BAV3 genome.

20. The method of any preceeding claims, wherein said first cell line is a nonhuman cell line.

21. The method of claim 20, wherein said first cell line has a bovine origin and wherein said first adenoviral helper vector is or derives from a BAV3 genome.

22. The method of any preceeding claim, wherein said first cell line is capable of complementing part or all of at least one defective function of said first or second or first and second helper(s).

23. The method of claim 22, wherein said first cell line is complementing the E1 function of said first or second or first and second adenoviral helper vector(s).

24. The method of any preceeding claims wherein said second cell line is of human origin.

25. The method of claim 24 wherein said second cell line is capable of complementing part or all of at least one defective function of said recombinant minimal vector.

26. The method of claim 25, wherein said second cell line is capable of complementing the Ad5 E1 function.

27. The method of claim 26, wherein said second cell line is selected among the group consisting of PER-C6 and 293.

28. The method of any preceeding claims, which comprises more than one amplification step, wherein said viral particles obtained in step (f) are used to reinfect said second cell line in the presence of fresh second adenoviral helper vector or virus.

29. The method of any preceeding claims, which further comprises a purification step of the viral particles obtained in step (f).

30. The method of any preceeding claims, wherein said viral particles obtained in step (f) are substantially helper-free.

31. An animal adenovirus genome having modified 5′ and 3′ ITRs and wherein said modification consists in the replacement of:
the penultimate 20 bp containing the core origin,
the penultimate 50 bp containing the entire origin of replication or
the entire ITRs of said animal adenovirus genome by the homologue sequences of the 5′ and 3′ ITRs of a human adenovirus genome.

32. A viral preparation obtained according to the method of any proceeding claims, wherein said viral preparation is substantially helper-free.

33. A host cell comprising a viral preparation according to claim 32.

34. A pharmaceutical composition comprising a viral preparation according to claim 32 or a host cell according to claim 33.

35. Use of a viral preparation according to claim 32 or a host cell according to claim 33 for the preparation of a medicament for the treatment of diseases by gene therapy or immunotherapy.
